(19) United States
(12) Patent Application Publication
(10) Pub. No.: US 2024/0277015 A1
(43) Pub. Date: Aug. 22, 2024

(54) FERMENTED FOOD PRODUCT, METHOD FOR PRODUCING FERMENTED FOOD PRODUCT, AND METHOD FOR IMPARTING TEXTURE AND SWEETNESS TO FERMENTED FOOD PRODUCT

(71) Applicants: Mizkan Holdings Co., Ltd., Aichi (JP); Mizkan Co., Ltd., Aichi (JP)

(72) Inventors: Hiroyuki Kakuda, Aichi (JP); Hiromichi Kawa, Aichi (JP); Hideyuki Ichise, Aichi (JP); Yoshitaka Morishita, Aichi (JP); Arisa Takiguchi, Aichi (JP)

(73) Assignees: Mizkan Holdings Co., Ltd., Aichi (JP); Mizkan Co., Ltd., Aichi (JP)

(21) Appl. No.: 18/583,430

(22) Filed: Feb. 21, 2024

Related U.S. Application Data
(63) Continuation of application No. PCT/JP2022/042530, filed on Nov. 16, 2022.

(30) Foreign Application Priority Data
Nov. 19, 2021 (JP) 2021-188996

(51) Int. Cl. A23L 11/50; A23L 31/00

(52) U.S. Cl. CPC A23L 11/50; A23L 31/00

(57) ABSTRACT

Provided is a fermented food having sweetness without depending on addition of saccharides. A fermented food obtained by fermenting a raw material with koji, in which the fermented food satisfies the following requirements: (a) the raw material is one or more kinds selected from the group consisting of beans, cereals, vegetables, and nuts and seeds; and (b) the salt content is 1000 mg or less per 100 g of the fermented food.

### TECHNICAL FIELD

The present invention relates to a fermented food, a method for producing a fermented food, a method for imparting texture and sweetness to a fermented food, and the like.

### BACKGROUND ART

Fermented foods typified by natto are widely consumed in Japan, and are rich in protein and high in nutritional value, and thus have become one of daily foods for Japanese. In addition, since bacteria used for fermentation and enzymes produced by the bacteria have a probiotic property such as an intestinal regulation effect or a prebiotic property, the fermented food is useful as a ready-to-eat food that contributes to health. Therefore, it is desired to develop a fermented food that can be taken in on a daily basis.

However, fermented foods of edible plants including beans and cereals have a problem that there is no fermented food having both sweetness without harshness and texture.

### CITATION LIST

### Patent Literature

PTL 1: JP2006-311836A

### SUMMARY OF INVENTION

### Technical Problem

Regarding a fermented food having sweetness, Patent Literature 1 proposes a method for producing a fermented food, in which beans other than soybean, and nuts and seeds are used as raw materials, and a saccharide is added after being cooled after fermentation by Bacillus subtilis natto. However, while consumer awareness of foods is increasing, the sweetness of foods themselves is regarded as important, but expression of sweetness due to addition of saccharides is not desirable. In addition, fermented foods of edible plants including beans and cereals have a problem that not only the surface is hard and has no taste, but also the texture and taste are monotonous and easy to get bored.

An object of the present invention is to provide a fermented food having a unique texture and sweetness of a vicinity of a surface and a central part of a food composition.

### Solution to Problem

As a result of intensive studies in view of the above problems, the present inventors have found that a fermented food obtained by fermenting a raw material with a powder having α-amylase activity, in which the fermented food satisfies the following requirements: (a) the raw material is one or more kinds selected from the group consisting of beans, cereals, vegetables, and nuts and seeds; and (b) the salt content is 1000 mg or less per 100 g of the fermented food. As a result of further research based on this finding, the present inventor has completed the present invention. That is, the present invention includes the following aspects.

### Item 1

A fermented food including a solid raw material and a powder having α-amylase activity, wherein the fermented food satisfies the following requirements:
(a) the raw material is one or more kinds of edible plants selected from the group consisting of beans, cereals, vegetables, and nuts and seeds; and
(b) the salt content is 1000 mg or less per 100 g of the fermented food, or 800 mg or less, or 600 mg or less, or 500 mg or less, or 400 mg or less, or 300 mg or less, or 200 mg or less, or 100 mg or less, or 50 mg or less, and the lower limit is 1 mg or more, or 2 mg or more, or 3 mg or more.

### Item 2

The fermented food according to the item regarding the fermented food, wherein the powder having α-amylase activity is a powder of a microorganism.

### Item 3

The fermented food according to any one of the preceding items regarding the fermented food, wherein the microorganism is koji.

### Item 4

The fermented food according to any one of the preceding items regarding the fermented food, wherein the koji is koji-made.

### Item 5

The fermented food according to any one of the preceding items regarding the fermented food, wherein the fermented food is obtained by fermenting the raw material with the koji.

### Item 6

The fermented food according to any one of the preceding items regarding the fermented food, further satisfying the following requirements that:
a mass ratio of the powder having α-amylase activity to the raw material is 1:3 or more, or 1:4 or more, or 1:5 or more, or 1:6 or more, and 1:10 or less, or 1:9 or less, or 1:8 or less, or 1:7 or less in mass ratio.

### Item 7

The fermented food according to any one of the preceding items regarding the fermented food, further satisfying the following requirements that:
d50 after sonication of the powder having α-amylase activity is 1000 μm or less, or 750 μm or less, or 500 μm or less, or 400 μm or less, or 350 μm or less, and 0.1 μm or more, or 1 μm or more, or 10 μm or more, or 25 μm or more, or 50 μm or more.

### Item 8

The fermented food according to any one of the preceding items regarding the fermented food, further satisfying the following requirements that:
the dry basis moisture content of the powder having α-amylase activity is 15% by mass or less, or 10% by mass or less, or 5% by mass or less, and 0.1% by mass or more, or 0.5% by mass or more, or 1% by mass or more.

### Item 9

The fermented food according to any one of the preceding items regarding the fermented food, further satisfying the following requirements that:
the α-amylase activity of the powder having α-amylase activity is 10 U/g or more, or 20 U/g or more, or 30 U/g or more, or 40 U/g or more, or 50 U/g or more, or 60 U/g or more, or 70 U/g or more, or 80 U/g or more, or 90 U/g or more, or 100 U/g or more, or 150 U/g or more, or 200 U/g or more, or 300 U/g or more, or 400 U/g or more, or 500 U/g or more, or 600 U/g or more, or 700 U/g or more, or 800 U/g or more, or 900 U/g or more, or 1000 U/g or more, or 1500 U/g or more, or 2000 U/g or more, and 100,000 U/g or less, or 80,000 U/g or less.

### Item 10

The fermented food according to any one of the preceding items regarding the fermented food, further satisfying the following requirements that:
the starch content in the raw material is 4% by mass or more, or 10% by mass or more, or 15% by mass or more, or 20% by mass or more, or 25% by mass or more, or 30% by mass or more, and 95% by mass or less, or 90% by mass or less, or 80% by mass or less, or 70% by mass or less, or 60% by mass or less, or 55% by mass or less.

### Item 11

The fermented food according to any one of the preceding items regarding the fermented food, further satisfying the following requirements that:
a dry basis moisture content of the raw material is 40% by mass or more, or 55% by mass or more, or 60% by mass or more and 95% by mass or less, or 90% by mass or less, or 85% by mass or less.

### Item 12

The fermented food according to any one of the preceding items regarding the fermented food, further satisfying the following requirements that:
a ratio of a dry basis moisture content of the powder having α-amylase activity to a dry basis moisture content of the raw material (the dry basis moisture content of the powder having α-amylase activity/the dry basis moisture content of the raw material) is 0.001 or more, or 0.01 or more, or 0.1 or more and 0.3 or less, or 0.2 or less.

### Item 13

The fermented food according to any one of the preceding items regarding the fermented food, wherein the beans are one or more kinds of selected from the group consisting of Pisum, Cicer, and Phaseolus.

### Item 14

The fermented food according to any one of the preceding items regarding the fermented food, wherein the cereals are one or more kinds of cereals selected from the group consisting of Oryza, Zea, Hordeum, and Triticeae.

### Item 15

The fermented food according to any one of the preceding items regarding the fermented food, wherein the cereals are miscellaneous grains.

### Item 16

The fermented food according to any one of the preceding items regarding the fermented food, wherein the miscellaneous grains are one or more kinds of miscellaneous grains selected from the group consisting of awa (millet), hie (Japanese barnyard millet), kibi (proso millet), sorghum, rye, oat, adlay, corn, buckwheat, amaranth, and quinoa.

### Item 17

The fermented food according to any one of the preceding items regarding the fermented food, wherein the vegetables are one or more kinds of vegetables selected from the group consisting of Solanum, Ipomoea, and Cucurbita.

### Item 18

The fermented food according to any one of the preceding items regarding the fermented food, wherein the nuts and seeds are one or more kinds of nuts and seeds selected from the group consisting of almond, Cannabis, linseed, perilla, cashew nut, pumpkin seed, catta, ginkgo nut, Japanese chestnut, walnut, poppy, coconut, sesame, Castanopsis, Japanese horsechestnut, Lotus seed, Trapa japonica, Pistachio, sunflower seed, brazil nut, hazelnut, pecan, macadamia nut, pine, and peanut.

### Item 19

The fermented food according to any one of the preceding items regarding the fermented food, wherein the potatoes are one or more kinds of potatoes selected from the group consisting of sweet potato, cassava, yacon, taro, Colocasia esculenta, Amorphophalus potato, Tacca leontopetaloides (Polynesian arrowroot), potato, purple sweet potato, Jerusalem artichoke, Katakuri, yam, Japanese yam, Chinese yam, and kudzu.

### Item 20

The fermented food according to any one of the preceding items regarding the fermented food, wherein the raw material is mushrooms.

### Item 21

The fermented food according to any one of the preceding items regarding the fermented food, wherein the raw material is fruits.

### Item 22

The fermented food according to any one of the preceding items regarding the fermented food, further satisfying the following requirements that:
the content of the soluble carbohydrate is 5% by mass or more, or 6% by mass or more, or 7% by mass or more, or 8% by mass or more, or 10% by mass or more and 40% by mass or less, or 19% by mass or less, or 18% by mass or less, or 17% by mass or less.

### Item 23

The fermented food according to any one of the preceding items regarding the fermented food, further satisfying the following requirements that:
the soluble carbohydrate is glucose and/or maltose.

### Item 24

The fermented food according to any one of the preceding items regarding the fermented food, further satisfying the following requirements that:
the glucose content is 5% by mass or more, 6% by mass or more, 7% by mass or more, or 8% by mass or more, or 12% by mass or more, or 13% by mass or more, and 30% by mass or less, or 19% by mass or less, or 18% by mass or less, or 17% by mass or less.

### Item 25

The fermented food according to any one of the preceding items regarding the fermented food, further satisfying the following requirements that:
the maltose content is 0.1% by mass or more, or 0.2% by mass or more, or 0.3% by mass or more, or 0.6% by mass or more, or 0.63% by mass or more, or 0.65% by mass or more, and is 0.7% by mass or more, and 5% by mass or less, or 3% by mass or less, or 2% by mass or less, or 1.5% by mass or less.

### Item 26

The fermented food according to any one of the preceding items regarding the fermented food, further satisfying the following requirements that:
the free arginine content is 0.5 mg or more, or 1.0 mg or more, or 5 mg or more, or 10 mg or more, or 50 mg or more, or 70 mg or more, or 90 mg or more, or 100 mg or more and 190 mg or less, or 185 mg or less, or 175 mg or less, or 160 mg or less per 100 g of the fermented food.

### Item 27

The fermented food according to any one of the preceding items regarding the fermented food, further satisfying the following requirements that:
the 2-phenylethyl alcohol content is 5 μg or more, or 5.5 μg or more, or 6 μg or more, or 7 μg or more, and 100 μg or less, or 80 μg or less, or 50 μg or less, or 30 μg or less per 1 kg of the fermented food.

### Item 28

The fermented food according to any one of the preceding items regarding the fermented food, further satisfying the following requirements that:

The 4-vinyl-2-methoxyphenol content is 20 μg or more, or 25 μg or more, or 30 μg or more, or 40 μg or more, and 230 μg or less, or 150 μg or less, or 100 μg or less, or 90 μg or less per 1 kg of the fermented food.

### Item 29

The fermented food according to any one of the preceding items regarding the fermented food, further satisfying the following requirements that:

The branched chain fatty acid content is 9.0 mg or less, or 8.0 mg or less, or 7.0 mg or less, or 6.0 mg or less, or 5.0 mg or less per 100 g of the fermented food.

### Item 30

The fermented food according to any one of the preceding items regarding the fermented food, further satisfying the following requirements that:

The raw material has a dietary fiber content of 1.0% by mass or more, or 1.5% by mass or more, or 2% by mass or more, or 9% by mass or more, or 12% by mass or more, or 15% by mass or more and 80% by mass or less, or 70% by mass or less, or 60% by mass or less.

### Item 31

The fermented food according to any one of the preceding items regarding the fermented food, further satisfying the following requirements that:
the raw material has a diameter or a major axis length of 4 mm or more, or 5 mm or more, or 6 mm or more, or 7 mm or more, or 8 mm or more, or 10 mm or more, and 50 mm or less, or 45 mm or less, or 40 mm or less, or 35 mm or less, or 30 mm or less, or 25 mm or less.

### Item 32

The fermented food according to any one of the preceding items regarding the fermented food, wherein the raw materials are fermented with the koji and the Bacillus subtilis natto.

### Item 33

The fermented food according to any one of the preceding items regarding the fermented food, further satisfying the following requirements that:
the viable cell count of Bacillus subtilis natto is 1.0×10⁶ cells/g or more, or 2.0×10⁵ cells/g or more, or 3.0×10⁵ cells/g or more, or 4.0×10⁵ cells/g or more and 1.0×10⁹ cells/g or less, or 8.0×10⁸ cells/g or less, or 6.0×10⁸ cells/g or less, or 4.0×10⁸ cells/g or less.

### Item 34

The fermented food according to any one of the preceding items regarding the fermented food, further satisfying the following requirements that:

The ratio (mass/surface area) of the mass of the coating with the powder having α-amylase activity to the surface area of the edible plant as a raw material is 0.03 g/100 cm² or more, or 0.1 g/100 cm² or more, or 0.15 g/100 cm² or more and 10.0 g/100 cm² or less, or 6.0 g/100 cm² or less, or 4.5 g/100 cm² or less, or 3.0 g/100 cm² or less.

### Item 35

The fermented food according to any one of the preceding items regarding the fermented food, further satisfying the following requirements that:
the coverage of the powder having α-amylase activity with respect to the surface area of the edible plant as a raw material is 50% or more, or 60% or more, or 70% or more, or 80% or more, or 90% or more, and 100% or less, or 95% or less.

### Item 36

The fermented food according to any one of the preceding items regarding the fermented food, further satisfying the following requirements that:
the starch content after the fermentation step is 70% by mass or less, or 60% by mass or less, or 55% by mass or less, and 5% by mass or more, or 10% by mass or more, or 15% by mass or more, or 20% by mass or more, or 25% by mass or more.

### Item 37

The fermented food according to any one of the preceding items regarding the fermented food, further satisfying the following requirements that:
a powder having an α-amylase activity is a koji powder that is koji-made, and the dry basis moisture content of the powder is 15% by mass or less, or 10% by mass or less, or 5% by mass or less, and 0.1% by mass or more, or 0.5% by mass or more, or 1% by mass or more, and d50 after sonication is 1000 μm or less, or 750 μm or less, or 500 μm or less, or 400 μm or less, or 350 μm or less, and 0.1 μm or more, or 1 μm or more, or 10 μm or more, or 25 μm or more, or 50 μm or more.

### Item 38

A method for producing a fermented food by adding a powder having an α-amylase activity to a solid raw material, the method satisfying all of the following steps (I) to (IV):
(I) a step of adjusting a composition satisfying the following conditions:
   (a) the raw material is one or more kinds of edible plants selected from the group consisting of beans, cereals, vegetables, and nuts and seeds; and
   (b) a salt content is 1000 mg or less per 100 g of the fermented food,
(II) a step of adding a powder having α-amylase activity and/or Bacillus subtilis natto to the composition of (1), and
(III) a step of fermenting the composition of (II).

### Item 39

The method according to any one of the preceding items regarding the method, further satisfying the following requirements that:
the powder having α-amylase activity is a powder of koji.

### Item 40

The method according to any one of the preceding items regarding the method, further satisfying the following requirements that:
the koji is koji-made.

### Item 41

The method any one of the preceding items regarding the method, further satisfying the following requirements that:
koji and Bacillus subtilis natto are added in the step (II).

### Item 42

The method any one of the preceding items regarding the method, further satisfying the following requirements that:
Bacillus subtilis natto is added to the composition obtained by mixing the composition of (1) and koji in the step (II).

### Item 43

The method any one of the preceding items regarding the method, further satisfying the following requirements that:
koji is added to the composition obtained by mixing the composition of (1) and Bacillus subtilis natto in the step (II).

### Item 44

The method any one of the preceding items regarding the method, wherein in the step (II), fermentation is performed at a product temperature of 30° C. or more and 60° C. or less for 5 hours or more, or 6 hours or more, or 7 hours or more, and 23 hours or less, or 22 hours or less, or 21 hours or less.

### Item 45

The method any one of the preceding items regarding the method, wherein in the step (II), the product temperature is maintained at a lower limit of 45° C. or more, or 46° C. or more, or 47° C. or more, and an upper limit of 54° C. or less, or 53.75° C. or less, or 53.5° C. or less for 4 hours or more, or 5 hours or more, or 8 hours or more, or 10 hours or more, and at an upper limit of the high-temperature fermentation time of 22 hours or less, or 20 hours or less, or 18 hours or less, or 16 hours or less.

### Item 46

The method any one of the preceding items regarding the method, further including the following step (IV):
(IV) a step of performing fermentation at a product temperature of 55° C. or more and 62° C. or less, or 57° C. or more and 61° C. or less, or 59° C. or more and 60.5° C. or less for 0.5 hours or more and 3 hours or less, or 1 hour or more and 2 hours or less after the step (III).

### Item 47

The method any one of the preceding items regarding the method, wherein in the step (II), the mass ratio of the powder having α-amylase activity to the raw material is 1:3 or more, or 1:4 or more, or 1:5 or more, or 1:6 or more, and 1:10 or less, or 1:9 or less, or 1:8 or less, or 1:7 or less.

### Item 48

The method any one of the preceding items regarding the method, further including the following step (V):

(V) a step of aging at a low temperature of 3° C. or more and less than 10° C., or 3° C. or more and less than 8ºC, or 3° C. or more and less than 6° C. for about 6 hours to 3 days, or 8 hours to 2 days, or 24 hours after completion of fermentation.

### Item 49

The method any one of the preceding items regarding the method, wherein in the step (II) and/or (IV), the starch content decrease rate (that is, the starch decreasing rate determined by “(% by mass of the starch before fermentation −% by mass of the starch after fermentation) % by mass of the starch before fermentation”) of the raw material is adjusted to 1% by mass or more, 2% by mass or more, 3% by mass or more, 4% by mass or more, 5% by mass or more, 6% by mass or more, 7% by mass or more, 8% by mass or more, 9% by mass or more, 10% by mass or more, 15% by mass or more, 20% by mass or more, 25% by mass or more, 30% by mass or more, 35% by mass or more, 40% by mass or more, 45% by mass or more, 50% by mass or more, 55% by mass or more, and 100% by mass or less, or 90% by mass or less.

### Item 50

The method any one of the preceding items regarding the method, wherein in the step (I) and/or (IV), the dietary fiber content decrease rate (that is, the dietary fiber decreasing rate determined by “(% by mass of the dietary fiber before fermentation −% by mass of the dietary fiber after fermentation)/% by mass of the dietary fiber before fermentation”) of the raw material is adjusted to 1% by mass or more, 2% by mass or more, 3% by mass or more, 4% by mass or more, 5% by mass or more, 6% by mass or more, 7% by mass or more, 8% by mass or more, 9% by mass or more, 10% by mass or more, 15% by mass or more, 20% by mass or more, 25% by mass or more, 30% by mass or more, 35% by mass or more, 40% by mass or more, 45% by mass or more, 50% by mass or more, 55% by mass or more, and 100% by mass or less, or 90% by mass or less.

### Item 51

The method any one of the preceding items regarding the method, wherein in the step (II) and/or (IV), the starch content is adjusted to 70% by mass or less, or 60% by mass or less, or 55% by mass or less, and 5% by mass or more, or 10% by mass or more, or 15% by mass or more, or 20% by mass or more, or 25% by mass or more.

### Item 52

The method any one of the preceding items regarding the method, further satisfying the following requirements that:
the powder having α-amylase activity in the step (II) has a dry basis moisture content of 15% by mass or less, or 13% by mass or less, or 10% by mass or less, or 8% by mass or less, and 0.1% by mass or more, or 0.3% by mass or more, or 0.6% by mass or more, or 1.0% by mass or more.

### Item 53

The method any one of the preceding items regarding the method, further satisfying the following requirements that:
d50 after sonication of the powder having α-amylase activity in the step (II) is 1000 μm or less, or 750 μm or less, or 500 μm or less, or 400 μm or less, or 350 μm or less, and 0.1 μm or more, or 1 μm or more, or 10 μm or more, or 25 μm or more, or 50 μm or more.

### Item 54

The method any one of the preceding items regarding the method, further satisfying the following requirements that:
the powder having α-amylase activity in the step (II) is retained at 4° C. or more, or 10° C. or more, or 15° C. or more, or 20° C. or more and 40° C. or less, or 30° C. or less, or 25° C. or less, for a storage time of 1 hour or more, or 3 hours or more, or 4 hours or more and 700 hours or less, or for 600 hours or less, or for 500 hours or less, or for 400 hours or less, or for 300 hours or less, or for 240 hours or less, or 168 hours or less.

### Item 55

The method any one of the preceding items regarding the method, further satisfying the following requirements that:
the raw material in the step (I) is heat-treated at 80° C. or more under the condition of a dry basis moisture content of 50% by mass or more.

### Item 56

The method any one of the preceding items regarding the method, further satisfying the following requirements that:
a powder having an α-amylase activity is a koji powder that is koji-made, and the dry basis moisture content of the powder is 15% by mass or less, or 10% by mass or less, or 5% by mass or less, and 0.1% by mass or more, or 0.5% by mass or more, or 1% by mass or more, and d50 after sonication is 1000 μm or less, or 750 μm or less, or 500 μm or less, or 400 μm or less, or 350 μm or less, and 0.1 μm or more, or 1 μm or more, or 10 μm or more, or 25 μm or more, or 50 μm or more.

### Item 57

A method for imparting a sweetness and a texture to a fermented food, wherein when a sweetness is imparted to a fermented food using beans, cereals, vegetables, and nuts and seeds as raw materials, koji is used, and a salt content is 1000 mg or less per 100 g of the food.

### Item 58

A food product including the fermented food according to any one of the preceding items regarding the fermented food.

### Advantageous Effects of Invention

According to the present invention, it is possible to provide a fermented food having a sweetness without depending on the addition of a saccharide and having a texture like al dente. This makes it possible to provide a fermented food that can be eaten on a daily basis.

### BRIEF DESCRIPTION OF DRAWINGS

FIG. 1 is a graph showing temporal changes in a control temperature (room temperature) and a product temperature of a fermentation chamber during fermentation of a sample in Examples and Reference Examples other than Reference Examples b3 and b4 of Test Example 2. More specifically, FIG. 1 is a graph showing a temporal change in the product temperature during fermentation in “a been fermented food containing Bacillus subtilis natto in which a chickpea maintained at room temperature of 37° C. is used as a raw material”, “a been fermented food containing Bacillus subtilis natto in which a soybean maintained at room temperature of 50° C. is used as a raw material”, and “a been fermented food containing Bacillus subtilis natto in which a chickpea maintained at room temperature of 50° C. is used as a raw material”.

### DESCRIPTION OF EMBODIMENTS

In the present specification, the expressions “containing” and “including” include the concepts of “containing”, “including”, “consisting essentially of”, and “consisting only of”.

### 1. Fermented Food

The present invention, in one aspect thereof, relates to a fermented food including a solid raw material and a powder having α-amylase activity, wherein the fermented food satisfies the following requirements: (a) the raw material is one or more kinds of edible plants selected from the group consisting of beans, cereals, vegetables, and nuts and seeds; and (b) a salt content is 1000 mg or less per 100 g of the fermented food (In the present specification, the fermented food may be referred to as “the fermented food of the present invention”.).

The “fermentation” in the present invention refers to a process in which a component in food is changed by an endogenous enzyme of a microorganism to produce an organic substance, and includes a fermentation process associated with growth of the microorganism and a fermentation process by an endogenous enzyme possessed by the microorganism. That is, the “fermentation product” in the present invention includes a metabolite produced along with growth of a microorganism and a reaction product produced by an endogenous enzyme possessed by the microorganism. When fermentation is performed using endogenous enzymes possessed by microorganisms, the enzyme may be in any state, and the enzyme may be in a state in which the endogenous enzyme possessed by the microorganism is isolated and generated, or may be in a state of an inactivated bacterium (typically, a dead bacterium) in a state in which the proliferation activity of the microorganism is lost but the endogenous enzyme maintains the activity.

This is described below.

### <Edible Plant (Raw Material)>

The fermented food of the present invention contains a solid edible plant as a food material serving as a raw material thereof. The kind of edible plants contains one or more kinds of edible plants selected from the group consisting of nuts and seeds, cereals, beans, vegetables, potatoes, mushrooms, and fruits. More preferably, two or more kinds, three or more kinds, or four or more kinds may be used. The upper limit thereof is not particularly limited, but may be 10 kinds or less. Among them, it is preferable to contain beans. As the edible plant, in addition to the vegetable food material (vegetables, potatoes, mushrooms, fruits, algae, cereals (particularly miscellaneous grains), nuts and seeds, and the like) described in the food group classification described in STANDARD TABLES OF FOOD COMPOSITION IN JAPAN—2015—(Seventh Revised Edition), a wild grass (plantain, western bracken fern, Japanese butterbur, mugwort, etc.) usually provided for food as vegetables can also be used.

The properties of the raw material are not particularly limited, and the solid state of the raw material is preferable because an al dente-like texture can be imparted to the fermented food. The solid raw material may be any material as long as the texture can be felt at the time of chewing, and specifically, the shape of the raw material (soybean) may be used as it is like soybean natto, or a raw material subjected to processing such as crushing or shredding like hikiwari natto (crushed natto) may be used. The term “processing” as used herein includes not only mechanical processing such as pulverization and cleavage but also chemical processing such as drying treatment and solution treatment.

### <Beans>

Examples of the beans include, but are not limited to, peas (for example, yellow peas, white peas, green peas, blue peas, and especially green peas, which are immature seeds that are harvested in their pods in an immature state and are characterized by the green appearance of the beans), chickpeas, azuki beans, kidney beans (Phaseolus: such as red kidney beans and white kidney beans), lentils, Mung beans, green soybeans, kidney bean, black bean, pinto beans, tora beans, lima beans, safflower beans, pigeon pea, Chowpea, fava beans, Lens culinaris, lentils, peanut, lupin beans, grass peas, Ceratonia siliqua (carob), Parkia speciosa, Parkia biglobosa, (Mexican) jumping beans, and the like. It should be noted that a person skilled in the art who handles the food and processed products of the food can naturally understand the classification of the edible plants including the beans used in the present invention. As an example, the judgment can be made more clearly by referring to STANDARD TABLES OF FOOD COMPOSITION IN JAPAN—2020—(Eighth Revised Edition). In addition, it is possible to determine whether or not a food material of which some edible portions (green soybeans, green peas, and the like) are handled as vegetables is beans by the state of the entire plant (soybeans, peas, etc.) combined with the non-edible portion (sheath or the like).

Among them, from the viewpoint of the starch content, the genus of the beans are particularly preferably Pisum such as peas, Cicer such as chickpeas, or Phaseolus such as kidney beans. Furthermore, the content of Pisum, Cicer, and Phaseolus may be a predetermined rate or more with respect to the entire fermented food, and more specifically, may be 5% by mass or more, 10% by mass or more, 15% by mass or more, 20% by mass or more, 25% by mass or more, 30% by mass or more, 40% by mass or more, 50% by mass or more, 60% by mass or more, 70% by mass or more, 80% by mass or more, 90% by mass or more, or 100% by mass with respect to the entire fermented food.

In addition, the content of Glycine may be a predetermined rate or less with respect to the entire fermented food, and more specifically, may be 50% by mass or less, 40% by mass or less, 30% by mass or less, 20% by mass or more, 10% by mass or less, 5% by mass or less, or 1% by mass or less with respect to the entire fermented food.

In addition, the content of Glycine may be a predetermined rate or less with respect to the entire beans, and more specifically, may be 80% by mass or less, 70% by mass or less, 60% by mass or less, 50% by mass or less, 40% by mass or less, 30% by mass or less, 20% by mass or more, 10% by mass or less, 5% by mass or less, 1% by mass or less, or 0% by mass with respect to the entire beans.

In addition, the content of starch derived from of Pisum, Cicer, and Phaseolus may be a predetermined rate or more with respect to the content of starch of the entire fermented food, and more specifically, may be 5% by mass or more, 10% by mass or more, 15% by mass or more, 20% by mass or more, 25% by mass or more, 30% by mass or more, 40% by mass or more, 50% by mass or more, 60% by mass or more, 70% by mass or more, 80% by mass or more, 90% by mass or more, or 100% of the content of starch of the entire fermented food.

In addition, the content of starch derived from Glycine soybean may be a predetermined rate or less with respect to the entire fermented food, and more specifically, may be 80% by mass or less, 70% by mass or less, 60% by mass or less, 50% by mass or less, 40% by mass or less, 30% by mass or less, 20% by mass or more, 10% by mass or less, 5% by mass or less, 1% by mass or less, or 0% by mass of the entire fermented food.

In one aspect of the present invention, from the viewpoint of obtaining a particularly favorable flavor (chestnut like flavor) and texture, examples thereof preferably include Cicer such as chickpeas and Pisum such as yellow peas, and more preferably include the chickpeas. For example, the main chickpea includes a large grain type (Kaburi) having a bean grain size of about 10 to 13 mm and a skin color of skin, and a small grain type (Desi) having a bean grain size of about 7 to 10 mm and a dark brown, and both of them can be used. In addition, chickpeas subjected to crushing processing according to crushed natto using hikiwari (crushed) soybeans can also be used. Among them, it is preferable to use a large grain type because an al dente-like texture can be easily obtained.

The “flavor” means a feeling of combining a taste and a scent felt when a food is eaten. Therefore, the “chestnut flavor” herein primarily means a sense combined with the taste and aroma felt when eating chestnuts of nuts and seeds cooked into steamed, boiled, roasted chestnuts, etc. (food) (“chestnut flavor”, that is, “chestnut like flavor” in a narrow sense). In addition to this “chestnut flavor,” it also includes a flavor that reminds one of “chestnut flavor,” or in other words, a deep flavor produced by the proper balance of saccharides and aromatic components created by fermentation, and is therefore called “chestnut like flavor”.

### <Cereals>

Examples of cereals include, but are not limited to, corn (sweet corn, etc.), barley (glutinous wheat etc.), wheat, sorghum, oat, triticale, rye, buckwheat, fonio, quinoa, hie (Japanese barnyard millet), awa, proso millet, giant corn, sugar cane, amaranth and the like.

Among them, cereals other than rice are preferable from the viewpoint of dietary fiber content. Particularly preferred examples thereof include Zea such as corn, Hordeum such as barley, and Triticeae such as wheat.

### <Miscellaneous Grains>

In the present invention, the term “miscellaneous grains” refers to cereals other than rice, wheat, and barley, which are main cereals, among the aforementioned cereals, and is a concept also including pseudo cereals other than so-called Poaceae cereals (Chenopodiaceae, Amaranthaceae). When miscellaneous grains are used in the fermented food of the present invention, the kind of miscellaneous grains to be used is not limited, but as an example, one or more kinds of miscellaneous grains selected from Poaceae, Chenopodiaceae, and Amaranthaceae are preferable, and Poaceae is more preferable. Specific examples thereof include, but are not limited to, awa (millet), hie (Japanese barnyard millet), kibi (proso millet), sorghum, rye, oat (grain oat), adlay, corn, buckwheat, amaranth, and quinoa.

Examples of the vegetables are preferably, but are not limited to, potatoes such as sweet potato, cassava, yacon, taro, Colocasia esculenta, Amorphophalus potato, Tacca leontopetaloides (Polynesian arrowroot), potato, purple sweet potato, Jerusalem artichoke, Katakuri, yam, Japanese yam, Chinese yam, and kudzu. In addition to the potatoes, for example, pumpkin, carrot, radish, rutabaga, persnip, turnip, black salsify, lotus root, beet (preferably, beet (beet root): a variety improved for eating beet root), kuwai (arrowhead), shallot, garlic, peanut, kale, onion, asparagus, udo, cabbage, lettuce, spinach, Komatsuna, Qing geng cai, Chinese chive, green onion, Nozawana, sweet coltsfoot, chard (swischard), Mizuna, tomato, eggplant, green pepper, cucumber, Japanese ginger, cauliflower, broccoli, edible chrysanthemum, bitter gourd, okra, artichoke, zucchini, sugar beet, tiger nuts, ginger, perilla, wasabi, paprika, herbs (Cresson, coriander, Chinese water spinach, celery, tarragon, chive, chervil, sage, thyme, laurel, parsley, mustard green (brown mustard), mugwort, basil, oregano, rosemary, peppermint, savory, lemongrass, dill, wasabi leaf, Japanese pepper leaf, stevia), bracken, pohole, bamboo shoot and the like.

Among them, preferable examples of the vegetables include Solanum (particularly, potatoes) such as potato, Ipomoea such as sweet potato, and Cucurbita such as pumpkin.

### <Potatoes>

Examples of the potatoes include, but are not limited to, a sweet potato, a cassava, yacon, taro, Colocasia esculenta, Amorphophalus potato, Tacca leontopetaloides (Polynesian arrowroot), a potato, a purple sweet potato, Jerusalem artichoke, Katakuri, a yam, a Japanese yam, a Chinese yam, and kudzu.

Examples of the nuts and seeds include, but are not limited to, ginkgo nut, almond, cashew nut, pecan (pecan), macadamia nut, pistachio, hazelnut, coconut, pine nut, sunflower seed, pumpkin seed, watermelon seed, castanopsis, walnut, Japanese Chestnut, sesame, coffee bean (coffee cherry seed), cacao bean (cacao nut seed), and Brazil nut.

Among them, examples of the nuts and seeds includes preferably ginkgo nut.

### <Mushrooms>

Examples of the mushrooms include, but are not limited to, Chinese mushroom, Tricholoma matsutake, wood ear mushroom, Grifola frondosa, samokoshikake (Polypore), oyster mushroom, oyster mushroom, eryngii, Flammulina veluptipes, brown beech mushroom, japanese oak mushroom, mushroom, Pholiota microspora, Suillus bovinus, Lactarius hatsudake, and Lactarius volemus.

### <Fruits>

Examples of fruits include, but are not limited to, acerola, avocado, apricot, strawberry, fig, plum, citrus (Iyokan, Unsu mandarin orange, orange, grapefruit, lime, lemon etc.), olive, oyster, kiwi, guava, coconut, pomegranate, watermelon, plum, cherry (cherry, black cherry, etc.), jujube, pineapple, haskap, banana, papaya, loquat, grapes, berry (blueberry, raspberry etc.), mango, mangosteen, melon, peach, and apple.

<α-amylase>

The fermented food in the present invention contains a powder of α-amylase (CAS No. 9000-90-2) having a predetermined or more activity. α-amylase is an enzyme that irregularly cleaves the α-1,4-bond of starch to produce a polysaccharide, maltose, and an oligosaccharide. The α-amylase may be contained in a food material such as an edible plant to be a raw material of the fermented food of the present invention, may be added separately from the food material, may be produced with the production of the fermented food of the present invention, or may be a combination thereof. More specifically, purified α-amylase may be used, a food raw material containing α-amylase may be used, and a microorganism having α-amylase activity may be used. However, it is preferable to use a microorganism having α-amylase activity, and it is preferable to use koji as a microorganism having α-amylase activity.

The α-amylase preferably has a predetermined activity, and may be, for example, 10 U/g or more and 100,000 U/g or less. The lower limit is not particularly limited, and is 10 U/g or more, or 20 U/g or more, or 30 U/g or more, or 40 U/g or more, or 50 U/g or more, or 60 U/g or more, or 70 U/g or more, or 80 U/g or more, or 90 U/g or more, or 100 U/g or more, or 150 U/g or more, or 200 U/g or more, or 300 U/g or more, or 400 U/g or more, or 500 U/g or more, or 600 U/g or more, or 700 U/g or more, or 800 U/g or more, or 900 U/g or more, or 1000 U/g or more, or 1500 U/g or more, or 2000 U/g or more. The upper limit thereof is not particularly limited, and is usually 100,000 U/g or less, and preferably 80,000 U/g or less. The activity of α-amylase is preferably 800 U/g or more and 3000 U/g or less, and particularly preferably 1000 U/g or more and 2000 U/g or less.

The α-amylase used in the fermented food of the present invention is preferably in the form of a powder (sometimes described as a powder having α-amylase activity). As a result, it is possible to obtain an effect of easily covering the edible plant as a raw material. The “powder” of the present invention is obtained by finely crushing a solid material, and specifically, the dry basis moisture content described later may be 15% by mass or less, and the d50 after sonication described later may be 1000 μm or less. As the powder having α-amylase activity, a pulverized purified α-amylase may be used, a pulverized food raw material containing α-amylase may be used, or a pulverized microorganism having α-amylase activity may be used, and it is preferable to use a pulverized microorganism having α-amylase activity, and it is preferable to use koji powder as the powder of the microorganism having α-amylase activity.

### <Koji>

The koji may be any koji. Koji is a generic term for fermentation products produced by steaming grains or beans and then adding spores of mold called seed koji to propagate.

As a raw material for the production of the koji, any general grains or beans can be used, but typical examples thereof include rice, wheat and barley, and soybean.

In addition, any mold can be used as the mold as the seed koji mold used for the production of the koji mold as long as it is a mold used for brewing general foods, and typically, a mold of Aspergillus can be used. More specifically, white koji mold (for example, Aspergillus awamori var. kawachii, Aspergillus luchuensis mut. kawachii, Aspergillus usamii mut. shirousamii, Aspergillus kawachii, and the like), black koji mold (for example, Aspergillus awamori and the like), yellow koji mold (Aspergillus oryzac), soy sauce koji mold (Aspergillus sojac), and the like can be used.

In addition, it is a requirement that the koji has an enzyme activity such as a saccharifying enzyme for saccharifying a polysaccharide such as starch contained in the raw material into a monosaccharide at the time of use, and the state of life or death of the seed koji mold does not matter. In the present invention, the “monosaccharide” is a carbohydrate having the simplest structure by hydrolysis. Examples of the monosaccharide include, but are not limited to, glucose, xylose, mannose, galactose, arabinose, lyxose, and fructose, but in the present invention, the monosaccharide is preferably the total of glucose and fructose, which are monosaccharides usually contained in a large amount in a vegetable food. The content of a monosaccharide can be determined by comparing the content of the monosaccharide with the content of a monosaccharide standard having a known concentration, using high performance liquid chromatography in accordance with the method for measuring “Available Carbohydrates (Glucose, Fructose, Galactose, Sucrose (Sucrose), Maltose, Lactose and Trehalose)” in “STANDARD TABLES OF FOOD COMPOSITION IN JAPAN—2015—(Seventh Revised Edition) Analysis Manual”.

The koji in the present invention may be a koji mold in a vegetative cell state (so-called inoculum) usually used in the production of bean koji, or may be a koji in a spore state in which the α-amylase activity is increased to a prodetermined rate or more by the koji-making, and it is preferable to use a koji in a spore state in which the α-amylase activity and/or the cellulase activity is increased to a prodetermined rate or more by the koji-making. It is preferable to increase the α-amylase activity and/or the cellulase activity because the effect of the present invention is easily exhibited. The koji-made koji is a state in which the steamed raw material is inoculated with koji mold, and then subjected to the following steps at 30 to 40° C. for 48 hours or more: hikikomi (process of spreading steamed rice in a koji room and dissipating heat)→tokomomi (process of mixing the seed koji so that it evenly adheres to the steamed rice)→kirikaeshi (process of stirring the steamed rice)→mori (process of separating steamed rice into boxes to make it easier to control temperature and humidity)→middle work (process of spreading steamed rice thinly and flatly)→finishing work (process of spreading steamed rice even thinner in order to dry it)→releasing the koji. That is, it is preferable to use not koji molds in a vegetative cell state (so-called inoculum) which are usually used in the production of bean koji but koji molds in a spore state in which the α-amylase activity and/or the cellulase activity is increased to a predetermined rate or more by koji-making. Cellulase activity can be measured using a method according to the soy sauce test method (1985 Japan Soy sauce Research Institute). The α-amylase activity can be measured, for example, by the following method.

### · Preparation of Enzyme Liquid:

10 mL of a 0.5% NaCl/10 mM acetic acid buffer (pH 5) was added to 1 g of the pulverized measurement sample, and the mixture was allowed to stand at 4° C. for 16 hours, then the mixture was shattered into a paste by treatment at 25000 rpm for 30 seconds using a homogenizer NS52 (manufactured by MICROTEC CO., LTD.), further allowed to stand at 4° C. for 16 hours, and then filtered with a filter paper (Qualitative filter paper No. 2, manufactured by ADVANTEC Corporation) to obtain an enzyme liquid.

### · Activity Measurement:

In a test tube, 2 mL of a 0.05% soluble starch (starch (soluble) CAS No. 9005-25-8, product code 195-03961, manufactured by FUJIFILM Wako Pure Chemical Corporation) is put, and the resulting mixture is allowed to stand at 37° C. for 10 minutes. Then, 0.25 mL of the enzyme liquid is added, and the resulting mixture is mixed. The mixture is allowed to stand at 37° C. for 30 minutes, and then 0.25 mL of 1 M HCl is added and mixed. Thereafter, 0.25 mL of a potassium iodide solution containing 0.05 mol/L of iodine (0.05 mol/L iodine solution: (product code 091-00475) manufactured by FUJIFILM Wako Pure Chemical Corporation) is added and mixed, 11.5 mL of water is added and diluted, and the absorbance at a wavelength of 660 nm is measured with a spectrophotometer (absorbance A). In addition, as a control, 2 mL of a 0.05% soluble starch is placed in a test tube, left to stand at 37° C. for 40 minutes, then 0.25 mL of 1 M HCl is added and mixed, then 0.25 mL of an enzyme liquid and 0.25 mL of a 0.05 mol/L iodine solution are added in this order, and the mixture is diluted by adding 11.5 mL of water, and then the absorbance at a wavelength of 660 nm is measured with a spectrophotometer (absorbance B). The iodine solution in the present invention refers to a diluted solution of a potassium iodate solution (In the present invention, it may be simply referred to as “0.05 mol/L iodine solution” or “0.05 mol/L iodine liquid”.) containing 0.05 mol/L of iodine, and unless otherwise specified, a mixed potassium iodate solution (“0.05 mol/L iodine solution (product code 091-00475)” manufactured by FUJIFILM Wako Pure Chemical Corporation) containing 93.7% by mass of water, 0.24 mol/L (4.0% by mass) of potassium iodide, and 0.05 mol/L (1.3% by mass) of iodine is diluted and used. In addition, by diluting the “0.05 mol/L iodine solution” 200 times with water, a “0.25 mM iodine solution” can be obtained.

· Enzyme activity unit (U/g):

The absorbance reduction rate C (%) of the measurement sample during the enzymatic reaction for 30 minutes is determined from the absorbance reduction rate of the enzyme reaction section (absorbance A) with respect to the comparative target section (absorbance B), that is, ({(absorbance B-absorbance A/absorbance B}×100(%)). The enzyme activity for reducing the absorbance by 10% per 10 minutes is defined as 1 unit (U), and the enzyme activity per 1 g of the measurement sample is determined from the absorbance reduction rate C (%) when the enzyme reaction is carried out for 30 minutes with a 0.25 mL of an enzyme liquid (sample content: 0.025 g) by the following formula.

$$Enzyme activity unit ( U / g ) = \{ C \times ( 10 / 30 ) \times ( 1 / 10 ) \} / 0.025 \qquad [ Mathematical formula 1 ]$$

In addition, it is preferable that the koji is a koji having a predetermined or more α-amylase activity (particularly, koji-made koji). Specifically, it is preferable to use one in which the amylase activity of the koji mold is a predetermined rate or more. Specifically, the enzyme activity for dry koji may be 10 U/g or more and 100,000 U/g or less. The lower limit is not particularly limited, and is 10 U/g or more, or 20 U/g or more, or 30 U/g or more, or 40 U/g or more, or 50 U/g or more, or 60 U/g or more, or 70 U/g or more, or 80 U/g or more, or 90 U/g or more, or 100 U/g or more, or 150 U/g or more, or 200 U/g or more, or 300 U/g or more, or 400 U/g or more, or 500 U/g or more, or 600 U/g or more, or 700 U/g or more, or 800 U/g or more, or 900 U/g or more, or 1000 U/g or more, or 1500 U/g or more, or 2000 U/g or more. The upper limit thereof is not particularly limited, and is usually 100,000 U/g or less, and preferably 80,000 U/g or less. The activity of α-amylase is preferably 800 U/g or more and 3000 U/g or less, and particularly preferably 1000 U/g or more and 2000 U/g or less.

In addition, it is preferable that the koji is a koji having a predetermined or more cellulase activity (particularly, koji-made koji). Specifically, it is preferable to use one in which the cellulase activity of the koji mold is a predetermined rate or more. Specifically, the enzyme activity against dry koji is preferably 3 U/g or more, particularly 4 U/g or more, particularly 5 U/g or more, further 6 U/g or more, or 7 U/g or more, or 8 U/g or more, or 9 U/g or more, or 10 U/g or more. The upper limit thereof is not particularly limited, and is usually 1000 U/g or less or 800 U/g or less.

In addition, it is preferable that the koji is a koji having a predetermined or more acid protease activity (particularly, koji-made koji). Specifically, it is preferable to use one in which the acid protease activity of the koji mold is a prodetermined rate or more. Specifically, it is preferable that the enzyme activity for dry koji is 1000 U/g or more, particularly 1500 U/g or more, particularly 2000 U/g or more, further 2500 U/g or more, or 3000 U/g or more, or 3500 U/g or more, or 4000 U/g or more, or 5000 U/g or more, or 6000 U/g or more, or 7000 U/g or more, or 8000 U/g or more. The upper limit thereof is not particularly limited, and is usually 100,000 U/g or less or 80,000 U/g or less. The acid protease activity can be measured using a National Tax Agency prodetermined analysis method (The Japan Brewing Society, Commentary on the 4th revised National Tax Agency prodetermined analysis method, p. 223, 2003).

Furthermore, it is preferable to perform the fermentation treatment with a koji having the α-amylase activity and/or cellulase activity described above on the solid surface of the raw material, and in particular, it is particularly preferable to use a koji (in particular, koji-made koji) satisfying both the definition related to the α-amylase activity and the definition related to the cellulase activity described above because it is possible to provide a fermented food having a preferable sweetness while imparting a good texture like al dente to the fermented food using the solid raw material. Although the principle thereof is not clear, it is considered that the koji adhering to the vicinity of the surface of the solid raw material imparts a good texture while decomposing the raw material components from the vicinity of the surface of the composition, and a reaction product localized in the vicinity of the surface of the raw material at a stage where decomposition proceeds to a certain level or more (particularly, a decomposition product by α-amylase), or an enzyme reaction is converged by consumption of moisture in the vicinity of the surface of the raw material by a metabolic reaction, so that a fermented food having a texture different between the vicinity of the surface and the inside is obtained.

In addition, it is preferable to use a koji (in particular, koji-making koji) that satisfies both the above-mentioned definition relating to α-amylase activity or the definition relating to cellulase activity and the definition relating to acid protease, and it is preferable to use a koji (in particular, koji-made koji) that satisfies all of the definition relating to α-amylase activity and the definition relating to cellulase activity and the definition relating to acid protease.

As shown in the requirement (b), the salt content in the fermented food of the present invention is 0 mg or more and 1000 mg or less, preferably 0 mg or more and 500 mg or less, more preferably 0 mg or more and 300 mg or less, further preferably 0 mg or more and 100 mg or less, and particularly preferably 0 mg or more and 50 mg or less per 100 g of the fermented food of the present invention. In one aspect of the present invention, the lower limit of the salt content is, for example, 1 mg or more, preferably 2 mg or more, and more preferably 3 mg or more, and the upper limit of the salt content is, for example, 1000 mg or less, preferably 800 mg or less, more preferably 600 mg or less, further preferably 500 mg or less, further more preferably 400 mg or less, especially preferably 300 mg or less, especially further preferably 200 mg or less, especially further more preferably 100 mg or less, and particularly preferably 50 mg or less.

When the salt content is 1000 mg or less per 100 g of the fermented food, good sweet taste is easily exhibited, which is preferable. The salt in the present invention refers to the total salt contained in the composition, that is, the total salt containing not only the salt blended at the time of preparing the composition but also the salt contained in the food raw material and other optional components.

The salt content can be measured as follows.

The salt content in the fermented food is calculated by a method in which the sodium ion content is measured after acidolysis of the fermented food and converted into salt content. Nitric acid is used for acidolysis, but it is convenient to use an apparatus in which a decomposition operation is automated. An example of such a device is a DigiPREP (manufactured by GL Sciences Inc.). For the measurement of the sodium ion content, any method can be used as long as any instrument that can perform measurements by a general atomic absorption method or ICP mass spectrometry. Examples of the device capable of performing the measurement by ICP mass spectrometry include ICP-MS 7700 (manufactured by Agilent Technologies).

The fermented food of the present invention preferably satisfies the requirement that the ratio of the powder having α-amylase activity to the raw material is 1:3 or more and 1:10 or less by mass ratio. As the powder having α-amylase activity, it is preferable to use a powder of koji (in particular, koji-made koji).

In the fermented food of the present invention, the ratio of the powder having α-amylase activity to the raw material (the raw material immediately before the powder having α-amylase activity is added) is more preferably 1:4 or more and 1:9 or less by mass ratio, further preferably 1:4 or more and 1:8 or less by mass ratio, and further more preferably 1:5 or more and 1:7 or less by mass ratio. As the powder having α-amylase activity, it is preferable to use a powder of koji (in particular, koji-made koji).

In the fermented food of the present invention, when the ratio of the powder having α-amylase activity to the raw material is in the above range, the good flavor of the fermented food is improved, and further (particularly when Bacillus subtilis natto is used), the flavor of natto can be suppressed. As the powder having α-amylase activity, it is preferable to use a powder of koji (in particular, koji-made koji).

Furthermore, from the viewpoint of efficiently decomposing the polysaccharide contained in the raw material to improve the taste, it is preferable to increase the surface area of contact between the powder having α-amylase activity and the solid raw material, and it is preferable to use the powder having α-amylase activity as the fermented food of the present invention. Use of the powder having α-amylase activity is preferable because the surface of the raw material can be coated with the powder having α-amylase activity, and the al dente-like texture can be imparted to the fermented food. The powder having α-amylase activity is preferably a powder of koji (particularly koji-made koji). In addition, when the solid raw material has a predetermined amount or more of starch, α-amylase in the powder effectively acts, and the effect of the present invention is easily exhibited, which is preferable. The raw material is preferably an edible plant containing 4% by mass or more of starch, and specific examples thereof include beans, cereals, vegetables, and nuts and seeds. Among them, beans are preferable, and peas such as Pisum, chickpeas such as Cicer, and kidney beans such as Phaseolus are particularly preferable.

Here, the al dente-like texture refers to a unique texture generated by a synergistic effect of the hardness of the central part and the gradient of hardness from the vicinity of the soft surface to the central part having hardness.

The fermented food of the present invention preferably satisfies the requirement that the dry basis moisture content of the powder having α-amylase activity (particularly koji-made koji powder) is 15% by mass or less. In the present invention, “% by mass” (sometimes referred to as w/w %) represents the mass of the target component in percentage with respect to the mass of the entire food.

The dry basis moisture content of the powder having α-amylase activity (in particular, the koji-made koji powder) is preferably 15% by mass or less, more preferably 10% by mass or less, and further preferably 5% by mass or less. The lower limit thereof is not particularly limited, and is, for example, 0.1% by mass or more, 0.5% by mass or more, or 1% by mass or more. The “dry basis moisture content” in the present invention means the rate of the total amount of the water amount derived from the raw material of the composition of the present invention and the separately added water amount to the total amount of the solid content. The numerical value is measured by heating to 90° C. by a vacuum heat drying method in accordance with STANDARD TABLES OF FOOD COMPOSITION IN JAPAN—2020—(Eighth Revised Edition). Specifically, an appropriate amount of sample is collected and weighed (W1) in a scale container (W0) having a constant mass in advance, placed in a reduced pressure electric constant temperature dryer adjusted to a prodetermined temperature (more specifically, 90° C.) at normal pressure in a state where the lid of the scale container is removed or the mouth is opened, the door is closed, the vacuum pump is operated, drying is performed at a predetermined degree of pressure reduction for a certain period of time, the vacuum pump is stopped, the pressure is returned to normal pressure by sending dry air, the scale container is taken out, the lid is closed, and the sample is cooled in a desiccator, and then the mass is measured. In this way, drying, cooling, and weighing (W2) are repeated until the weight reaches a constant mass, and the dry basis moisture content (dry basis moisture content) (% by mass) is determined by the following calculation formula

$$Water content ( dry basis moisture content ) ( \% by mass ) = ( {W}_{1} - {W}_{2} ) / ( {W}_{2} - {W}_{0} ) \times 100 \qquad [ Mathematical formula 2 ]$$

(In the formula, W₀ is the mass (g) of the scale container with constant mass, W₁ is the mass (g) of the scale container containing the sample before drying, and W2 is the mass (g) of the scale container containing the sample after drying.)

By setting the dry basis moisture content of the powder having α-amylase activity within the above range, there is an advantage that the enzyme acts in a moisture-containing raw material to promote the decomposition of starch and increase the sweetness. In addition, when the dry basis moisture content of the koji is less than the above-mentioned upper limit value range, the starch decomposition of the koji itself does not proceed, and the action on the raw material is increased, so that the sweetness of the fermented food is increased, which is preferable. Furthermore, when the dry basis moisture content of the powder having α-amylase activity is within the above range, the surface of the raw material is easily coated with the powder having α-amylase activity, and the surface of the fermented food is softened to easily obtain an al dente-like texture, which is preferable.

The powder having α-amylase activity (in particular, koji-made koji powder) used in the fermented food of the present invention preferably has a size within a prodetermined range. Specifically, it is preferable that d50 (which may be simply referred to as “average particle size” in the present invention) after sonication of the powder having α-amylase activity is 1000 μm or less. In particular, the thickness is preferably 750 μm or less, more preferably 500 μm or less, particularly preferably 400 μm or less, particularly preferably 350 μm or less. The lower limit is not particularly limited, but is preferably 0.1 μm or more, or 1 μm or more, more preferably 10 μm or more, or 25 μm or more, and further preferably 50 μm or more from the viewpoint of industrial convenience. The particle size after the sonication can be measured using ethanol as a solvent by the procedure described later. In addition, it is preferable that the powdery koji mold is in the form of a complex and the d50 after the sonication is 500 μm or less and 50 μm or more, or 750 μm or less and 25 μm or more, particularly 500 μm or less and 50 μm or more because the powder has an α-amylase activity that adheres to the surface of the solid raw material and easily reacts.

After sonication of the powder having α-amylase activity, d50 can be adjusted to the above range using a sieve with an appropriate opening size. Specifically, by passing through the mesh of the opening size corresponding to the upper limit size of the sonication d50 and optionally collecting the koji powder on the mesh of the opening size corresponding to the lower limit size, it is possible to obtain a powder in which the d50 after the sonication has α-amylase activity within a certain range. By setting d50 after the sonication within the above range, the koji mold penetrates into the raw material, and a fermented food which is sweeter toward the center can be obtained. In addition, when the powder having α-amylase activity enters the gap of the raw material, the surface of the raw material can be coated with the powder having α-amylase activity, and a fermented food having a softer al dente-like texture can be obtained. When d50 after the sonication is within the above range, the powder having α-amylase activity can more favorably cover the surface of the raw material, and a sweetness or an al dente-like texture can be more favorably imparted. In particular, when d50 after the sonication of the koji is less than the above upper limit value, the surface of the raw material is more easily covered with koji, and the al dente-like texture can be more favorably imparted to the fermented food, which is preferable.

In the present invention, “mesh on” refers to a powder fraction having α-amylase activity that remains on a sieve of a specific size, and “mesh pass” refers to a powder fraction having α-amylase activity that passes through a sieve of a specific size. The content of each fraction is measured by fractionating the koji powder with sieves having different opening sizes. For example, “50 mesh on” means a powder fraction having α-amylase activity that remains on a 50 mesh sieve, and “0.1 mesh pass and 50 mesh on” means a powder fraction having α-amylase activity that passes through a 0.1 mesh sieve and remains on a 50 mesh sieve. The “mesh” in the present invention is a unit representing the density of meshes such as a wire mesh, a sieve, and a filter, and represents the number of meshes per inch. That is, for example, “1 mesh on (pass)” means the koji powder fraction that remains (passes) on a sieve with an opening size of 2.50 cm, “0.1 mesh on (pass)” means the powder fraction having α-amylase activity that remains (passes) on a sieve with an opening size of 25.0 cm, and “50 mesh on (pass)” means the powder fraction having α-amylase activity that remains (passes) on a sieve with an opening size of 300 micrometers.

Specifically, for the thickness of the wire of the mesh on and the spacing between the openings, the numerical values specified in U.S.A. Standard Testing Sieves ASTM Specifications E 11-04 (for example, 50 mesh corresponds to “No. 50” defined in “Alternative” in Nominal Dimensions, Permissible Variation for Wire Cloth of Standard Testing Sieves (U.S.A.) Standard Series in the same document, and 1 mesh corresponds to “1.00”) or similar numerical values were adopted, and the size can be measured by evenly spreading 100 g of a sample (20° C.) containing the koji powder to be measured on a sieve stacked in order from a sieve with a large opening size to a sieve with a small opening size stepwise, and processing the sample until the fraction mass on each sieve becomes constant while vibrating the sample with a load to the extent that the composition size does not change.

The size of the powder fraction having α-amylase activity of the present invention can be, for example, in the range of 200 mesh on and 0.1 mesh pass. Specifically, the lower limit thereof is preferably 200 mesh on, 140 mesh on, 100 mesh on, 60 mesh on, or 50 mesh on. On the other hand, the upper limit of the size of the powder having α-amylase activity of the present invention is not particularly limited, and is usually 0.1 mesh pass, particularly preferably 0.5 mesh pass, and further preferably 1 mesh pass.

The term “d50 after sonication” in the present invention refers to a particle size at which, when a particle size distribution obtained by measurement using a laser diffraction particle size distribution analyzer and ethanol as a measurement solvent is divided into two particle sizes from a certain particle size, the cumulative value of particle frequency % on the larger side and that on the smaller side are equal, and is also denoted as “d50”. The particle size in the present invention is all measured on a volume basis, and when not particularly limited, the measured value of the particle size represents a result obtained by analyzing a sample after sonication. In the present invention, unless otherwise specified, “sonication” refers to a treatment of applying an ultrasonic wave having a frequency of 30 kHz to a measurement sample in a measurement solvent at an output of 40 W for 3 minutes.

In one aspect of the present invention, it is preferable that a powder having α-amylase activity is a powder of koji-made koji, the powder has a dry basis moisture content of 15% by mass or less, or 10% by mass or less, or 5% by mass or less, and 0.1% by mass or more, or 0.5% by mass or more, or 1% by mass or more, and d50 after sonication of the powder is 1000 μm or less, or 750 μm or less, or 500 μm or less, or 400 μm or less, or 350 μm or less, and 0.1 μm or more, or 1 μm or more, or 10 μm or more, or 25 μm or more, or 50 μm or more. This makes it possible to provide a fermented food having an al dente-like texture and sweetness.

In the present invention, since it is difficult to measure the surface area of the edible plant as a raw material, the surface area was measured as the surface area (cm²) of the virtual rectangular parallelepiped. Specifically, the long diameter (cm), short diameter (cm), and thickness (cm) of the edible plant as a raw material were measured, and the surface area of the virtual rectangular parallelepiped was obtained by the calculation formula: surface arca=(long diameter×(short diameter×2+thickness×2)+short diameter×thickness×2)/100. It is to be noted that the volume of the edible plant as a raw material in the present invention is preferably 1 mm³ or more and 20 mm³ or less from the viewpoint of its eating property.

In the present invention, the ratio (mass/surface area) of the mass of the coating with the powder having α-amylase activity (in particular, koji-made koji powder) to the surface area of the edible plant as a raw material may be usually 0.03 g/100 cm² or more, but is preferably 0.1 g/100 cm² or more, and particularly more preferably 0.15 g/100 cm² or more from the viewpoint of imparting an al dente-like texture. On the other hand, the upper limit thereof is not particularly limited, and is usually preferably 10.0 g/100 cm² or less, more preferably 6.0 g/100 cm² or less, further preferably 4.5 g/100 cm² or less, and most preferably 3.0 g/100 cm² or less from the viewpoint of reducing the loss of the raw material.

In addition, the coverage of the powder having α-amylase activity (in particular, koji-made koji powder) with respect to the surface area of the edible plant as a raw material may be usually 50% or more, and is preferably 60% or more, more preferably 70% or more, further preferably 80% or more, and most preferably 90% or more from the viewpoint of imparting an al dente-like texture. On the other hand, the upper limit thereof is not particularly limited, and may be 100% or less or 95% or less. The coverage can be determined by measuring the surface area to which the powder having α-amylase activity adheres by the above-described method and calculating the coverage=(the surface area of the edible plant as the raw material to which the powder having an α-amylase activity is adhered/the surface area of the edible plant as the raw material)×100.

### <Starch>

The fermented food of the present invention preferably satisfies the requirement that the starch content of the raw material is 4% by mass or more.

The starch content (% by mass) of the raw material refers to the starch content in the raw material before water absorption treatment. When the raw material is not subjected to a water absorption treatment, it represents the starch content in the raw material before inoculating koji. The starch content can be measured according to AOAC method 996.11 (AOAC, 2005), for example, in accordance with the method in STANDARD TABLES OF FOOD COMPOSITION IN JAPAN—2020—(Eighth Revised Edition). The judgment can be made more clearly by referring to the item “starch” in STANDARD TABLES OF FOOD COMPOSITION IN JAPAN—2020—(Eighth Revised Edition).

The starch content of the raw material is more preferably 10% by mass or more, further preferably 15% by mass or more, further more preferably 20% by mass or more, especially preferably 25% by mass or more, and particularly preferably 30% by mass or more. The upper limit of the content is not particularly limited, and is, for example, 70 mass or less, preferably 60% by mass or less, and more preferably 55% by mass or less. When the starch content of the raw material exceeds the lower limit of the above range, there is an advantage that the water retainability of the raw material is enhanced and the texture of the fermented food is preferred.

In addition, it is preferable that the starch content of the raw material is reduced by a predetermined rate or more by fermentation using the koji in the present invention. Specifically, the starch content decreasing rate (that is, the starch decreasing rate determined by “(% by mass of starch before fermentation−% by mass of starch after fermentation) % by mass of starch before fermentation”) before and after fermentation is 1% by mass or more, particularly 2% by mass or more, more particularly 3% by mass or more, particularly 4% by mass or more, or 5% by mass or more, or 6% by mass or more, or 7% by mass or more, or 8% by mass or more, or 9% by mass or more, or 10% by mass or more, or 15% by mass or more, or 20% by mass or more, or 25% by mass or more, or 30% by mass or more, or 35% by mass or more, or 40% by mass or more, or 45% by mass or more, or 50% by mass or more, or 55% by mass or more. The upper limit of the decreasing rate is not particularly limited, and is usually 100% by mass or less or 90% by mass or less. In particular, it is considered that the starch in the vicinity of the surface of the solid raw material is decomposed to produce a fermented food having a different texture between the vicinity of the surface of the composition and the inside of the composition.

The starch content of the fermented food after the fermentation step is more preferably 5% by mass or more, further preferably 10% by mass or more, further more preferably 15% by mass or more, especially preferably 20% by mass or more, and particularly preferably 25% by mass or more. The upper limit of the content is not particularly limited, and is, for example, 70 mass or less, preferably 60% by mass or less, and more preferably 55% by mass or less.

When the starch content of the raw material is within the above range, there is an advantage that sweetness can be imparted to the fermented food.

The starch content (% by mass) of the fermented food can be measured according to AOAC method 996.11 (AOAC, 2005), for example, in accordance with the method in STANDARD TABLES OF FOOD COMPOSITION IN JAPAN—2020—(Eighth Revised Edition).

The fermented food of the present invention preferably satisfies the requirement that the raw material has a dietary fiber content of 1% by mass or more.

The dietary fiber content (% by mass) of the raw material refers to the dietary fiber content before water absorption treatment. When the raw material is not subjected to water absorption treatment, it refers to the content of dietary fiber in the raw material before inoculating koji. For example, it can be measured by an enzyme-weight method (Prosky method). Furthermore, “dietary fiber” can be more clearly determined by referring to the item of “total amount of dietary fiber” in STANDARD TABLES OF FOOD COMPOSITION IN JAPAN—2020-(Eighth Revised Edition).

The dietary fiber content of the raw material is more preferably 1.5% by mass or more, further preferably 2% by mass or more, further more preferably 9% by mass or more, especially preferably 12% by mass or more, and particularly preferably 15% by mass or more. The upper limit thereof is not particularly limited, and may be, for example, 80% by mass or less, preferably 70% by mass or less, and more preferably 60% by mass or less.

By setting the dietary fiber content of the raw material within the above range, a better texture can be imparted to the fermented food. In particular, when the dietary fiber content exceeds the lower limit of the above range, the koji decomposes the dietary fiber from the surface of the raw material, so that a texture having a gradient of hardness from the surface to the central part of the fermented food is obtained, which is preferable. In a preferred aspect of the present invention, an al dente-like texture can be imparted to the fermented food.

In addition, it is preferable that the dietary fiber content of the raw material is reduced by a predetermined rate or more by fermentation using the koji in the present invention. Specifically, the dietary fiber content decreasing rate (that is, the dietary fiber content decreasing rate determined by “(% by mass of dietary fiber content before fermentation−% by mass of dietary fiber content after fermentation)/% by mass of dietary fiber content before fermentation”) before and after fermentation is 1% by mass or more, particularly 2% by mass or more, more particularly 3% by mass or more, particularly 4% by mass or more, or 5% by mass or more, or 6% by mass or more, or 7% by mass or more, or 8% by mass or more, or 9% by mass or more, or 10% by mass or more, or 15% by mass or more, or 20% by mass or more, or 25% by mass or more, or 30% by mass or more, or 35% by mass or more, or 40% by mass or more, or 45% by mass or more, or 50% by mass or more, or 55% by mass or more. The upper limit of the decreasing rate is not particularly limited, and is usually 100% by mass or less or 90% by mass or less. In particular, it is considered that the insoluble dietary fiber in the vicinity of the surface of the solid raw material is decomposed to produce a fermented food having a different texture between the vicinity of the surface of the composition and the inside of the composition.

The dietary fiber content in the fermented food after the fermentation step is more preferably 5% by mass or more, further preferably 10% by mass or more, further more preferably 15% by mass or more, especially preferably 20% by mass or more, and particularly preferably 25% by mass or more. The upper limit of the content is not particularly limited, and is, for example, 70 mass or less, preferably 60% by mass or less, and more preferably 55% by mass or less.

By setting the dietary fiber content of the raw material within the above range, it is possible to obtain an advantage that an al dente-like texture can be imparted to the fermented food.

The hardness of the fermented food can be analyzed by, for example, a texture test (UD method) using a creepmeter. The creep meter is not particularly limited, and for example, RE2-33005 C (manufactured by YAMADEN co., ltd.) and an automatic analyzer (CA-0035, manufactured by YAMADEN co., ltd.) can be used.

The fermented food of the present invention preferably satisfies the requirement that the diameter of the raw material or the length of the major axis is 3 mm or more. The “major axis” of the raw material in the present invention represents the length in the long-side direction of the virtual rectangular parallelepiped having the minimum volume in which the composition is inscribed. When there is a plurality of“major axes” of the raw material, an arbitrary direction can be adopted. For example, in a spherical raw material, the diameter thereof can be set to the major axis.

The diameter or the length of the major axis of the raw material represents the diameter or the length of the major axis of the raw material before the water absorption treatment. When the raw material is not subjected to a water absorption treatment, it represents the diameter or the length of the major axis of the raw material before inoculating koji. As the raw material in the present invention, the shape of the raw material may be used as it is like soybean natto, or may be appropriately processed and used so as to satisfy the above range.

The diameter or the length of the major axis of the raw material is preferably 4 mm or more, more preferably 5 mm or more, further preferably 6 mm or more, further more preferably 7 mm or more, especially preferably 8 mm or more, and particularly preferably 10 mm or more. The upper limit of the content is not particularly limited, and is, for example, 50 mm or less, preferably 45 mm or less, more preferably 40 mm or less, further preferably 35 mm or less, further more preferably 30 mm or less, and especially preferably 25 m or less.

By setting the diameter of the raw material or the length of the major axis within the above range, it is possible to more favorably impart an al dente-like texture to the fermented food. In particular, when the size of the raw material exceeds the lower limit of the above range, the central part of the fermented food becomes harder than the surface part, and a texture having a gradient of hardness from the surface to the central part is obtained, which is preferable.

The fermented food of the present invention is preferably fermented using koji and Bacillus subtilis natto, and preferably satisfies the requirement that the viable cell count of Bacillus subtilis natto is 1.0×10⁵ cells/g or more and 1.0×10⁹ cells/g or less. The umami and richness can be imparted to the fermented food by amino acids, fatty acids, aromatic components, and the like produced by fermentation of Bacillus subtilis natto. In a preferred aspect of the present invention, a fermented food in which a fermentation odor is suppressed and which has a preferred flavor can be provided by allowing koji and Bacillus subtilis natto coexist and performing fermentation. In a further preferred aspect of the present invention, a novel chestnut like flavor can be imparted to the fermented food by allowing the koji and Bacillus subtilis natto to coexist and ferment the fermented food.

Note that “10^{N}” in the present specification represents the N-th power of 10, and for example, “1.0×10⁵” represents “100,000”.

The viable cell count of Bacillus subtilis natto is more preferably 2.0×10⁸ cells/g or more and 8.0×10⁸ cells/g or less, further preferably 3.0×10⁶ cells/g or more and 6.0×10⁸ cells/g or less, and further more preferably 4.0×10″ cells/g or more and 4.0×10⁸ cells/g or less.

When the viable cell count of Bacillus subtilis natto satisfies the above range, the effect of Bacillus subtilis natto on the health function can be sufficiently exhibited, and the degree of quality deterioration due to secondary fermentation or the like caused by Bacillus subtilis natto can be suppressed.

The viable cell count of Bacillus subtilis natto can be measured as follows.

The viable cell count in the fermented food can be measured by, for example, a method in which a diluted solution obtained by diluting a natto suspension is cultured on an agar medium to measure the number of colonies. The number of appearance of colonies other than Bacillus subtilis natto can be suppressed by shortening the culture time (37° C., 18 hours) as compared with the measurement of normal viable cells (about 37° C., 48 hours). For example, the fermented food natto is put in a bag with a filter attached to a paddle type blender “Stomacher (registered trademark)”, and a phosphate buffer solution is injected. The fermented food natto is shaken with the above Stomacher, and then diluted with the phosphate buffer. After that, the fermented food natto is mixed with a standard agar medium (manufactured by atect Corp.), and then cultured at 37° C. for 18 hours. It can be calculated from the number of colonies appearing therein.

The fermented food of the present invention preferably satisfies the requirement that the soluble carbohydrate content is 5% by mass or more and 30% by mass or less.

### <Soluble Carbohydrate>

In the present invention, the “soluble carbohydrate” is a carbohydrate soluble in water, and is a generic term for monosaccharides and oligosaccharides (saccharides to which about 2 to 10 monosaccharides are bonded). Therefore, starch, which is a component to which much more sugar is bound, is not included in the concept. The soluble carbohydrate content can be determined by summing each measurement value determined by comparing the content comparison with monosaccharides or oligosaccharides (2 to 10 sugars) standard products whose concentrations are known, using high performance liquid chromatography in accordance with the measurement method of “Available Carbohydrates (glucose, fructose, galactose, sucrose, maltose, lactose, and trehalose)” in “STANDARD TABLES OF FOOD COMPOSITION IN JAPAN—2015—(Seventh Revised Edition) Analysis Manual”. More specifically, the soluble carbohydrate in the present invention is preferably glucose and/or maltose. In the present specification, the soluble carbohydrate content may be referred to as a sugar content.

The fermented food of the present invention preferably has a glucose content of 5% by mass or more and 30% by mass or less. The content is more preferably 6% by mass or more and 19% by mass or less, further preferably 7% by mass or more and 18% by mass or less, and further more preferably 8% by mass or more and 17% by mass or less. Among these ranges, the content is preferably 10% by mass or more and 17% by mass or less, more preferably 12% by mass or more and 17% by mass or less, and further preferably 13% by mass or more and 17% by mass or less.

When the glucose content satisfies the above range, it is possible to express a certain level or more of sweetness while suppressing sticky taste due to too strong sweetness, and it is possible to achieve a good flavor balance with an aromatic component generated by fermentation.

The fermented food of the present invention preferably satisfies the requirement that the maltose content is 0.1% by mass or more and 5% by mass or less.

The maltose content is more preferably 0.63% by mass or more and 3% by mass or less, further preferably 0.65% by mass or more and 2% by mass or less, and further more preferably 0.7% by mass or more and 1.5% by mass or less. The lower limit of the content is 0.1% by mass or more, preferably 0.2% by mass or more, more preferably 0.3% by mass or more, further preferably 0.6% by mass or more, further more preferably 0.63% by mass or more, especially preferably 0.65% by mass or more, and especially further preferably 0.7% by mass or more. The upper limit of the content is 5% by mass or less, preferably 3% by mass or less, more preferably 2% by mass or less, and further preferably 1.5% by mass or less.

When the maltose content satisfies the above range, a certain level or more of the maltose specific flavor can be expressed while suppressing the maltose specific flavor from being too strong and sticky, so that the flavor balance with the aromatic components generated by fermentation can be improved.

The glucose content and the maltose content can be measured as follows.

The analysis of various saccharides is calculated by a method in which an extract obtained by extracting a fermented food with 50% ethanol is subjected to measurement by HPLC and detected by a differential refraction system. The instrument to be used is not particularly limited, and examples thereof include Prominence (manufactured by Shimadzu Corporation) as an HPLC system, HILICpak VG-50 4E HPLC (manufactured by Showa Denko K.K.) as a measurement column, and Shodex RI-201H (manufactured by Showa Denko K.K.) as a differential refractometer.

The fermented food of the present invention preferably satisfies the requirement that the free arginine content is 0.5 mg or more and 190 mg or less per 100 g of the fermented food.

The lower limit of the free arginine content is preferably 1.0 mg or more, more preferably 5 mg or more, further preferably 10 mg or more, further more preferably 50 mg or more, especially preferably 70 mg or more, especially further preferably 90 mg or more and particularly preferably 100 mg or more per 100 g of the fermented food. On the other hand, the upper limit thereof is preferably 185 mg or less, more preferably 175 mg or less, and further more preferably 160 mg or less per 100 g of the fermented food.

When the free arginine content satisfies the above range, slight bitterness can be felt while suppressing excessive bitterness.

The free arginine content can be measured as follows.

In the analysis of the amino acid composition, an extract obtained by extracting a fermented food sample with 50% ethanol is separated by ion exchange chromatography, then reacted with a ninhydrin reagent, and detected by a visible absorption detector. In the above measurement, a method using a fully automatic amino acid measuring machine capable of automatically performing a series of operations is simple, and examples of the fully automatic amino acid measuring machine include JLC-500/V2 (manufactured by JEOL Ltd.).

The fermented food of the present invention preferably satisfies the requirement that the content of 2-phenylethyl alcohol is 5 μg or more and 100 μg or less per 1 kg of the fermented food.

The content of 2-phenylethyl alcohol is more preferably 5.5 μg or more and 80 μg or less per 1 kg of the fermented food, further preferably 6 μg or more and 50 μg or less per 1 kg of the fermented food, and further more preferably 7 μg or more and 30 μg or less per 1 kg of the fermented food.

When the content of 2-phenylethyl alcohol satisfies the above range, an appropriate sweet flavor of a chestnut can be felt.

The fermented food of the present invention preferably satisfies the requirement that the content of 4 vinyl-2-methoxyphenol is 20 μg or more and 230 μg or less per 1 kg of the fermented food.

The content of 4-vinyl-2-methoxyphenol is more preferably 25 μg or more and 150 μg or less per 1 kg of the fermented food, further preferably 30 μg or more and 100 μg or less per 1 kg of the fermented food, and further more preferably 40 μg or more and 90 μg or less per 1 kg of the fermented food.

When the content of 4-vinyl-2-methoxyphenol satisfies the above range, an appropriate fermentation flavor can be felt.

The content of 4-vinyl-2-methoxyphenol and the content of 2-phenylethyl alcohol can be measured as follows.

Measurement of 4-vinyl-2-methoxyphenol and 2-phenylethyl alcohol is performed by a method of injecting them into GC-MS by a dynamic headspace method. Examples of the instrument to be used include a one-dimensional and two-dimensional switching GC-MS (GC section: LTM series II connected to HP7890 Series GC System (both manufactured by Agilent Technologies), injection port: TDU2/CIS4 (manufactured by GERSTEL K. K.), autosampler: MPS (manufactured by GERSTEL K. K.) manufactured by GERSTEL K. K.

The fermented food of the present invention preferably satisfies the requirement that the branched chain fatty acid content is 9.0 mg or less per 100 g of the fermented food.

The branched chain fatty acid content is more preferably 8.0 mg or less per 100 g of the fermented food, further preferably 7.0 mg or less per 100 g of the fermented food, further more preferably 6.0 mg or less per 100 g of the fermented food, and especially preferably 5.0 mg or less per 100 g of the fermented food.

When the branched chain fatty acid content satisfies the above range, an unpalatable flavor (particularly a natto odor) can be suppressed.

The branched chain fatty acid content can be measured using, for example, high performance liquid chromatography (HPLC) and a conductivity detector.

The form of the fermented food of the present invention is preferably a solid form in the case of a fermented food fermented only with koji. More specifically, for example, a form in which the shape of the raw material remains as it is, such as soybean natto, a form in which the raw material is crushed, such as crushed natto, a form in which the raw material is shattered, a form in which these are dried, and the like can be used. Since the fermented food of the present invention has good texture and sweetness, it is suitable for eating as it is after fermentation.

In the case of a fermented food fermented by Bacillus subtilis natto and koji mold, the form thereof is not particularly limited. For example, it can be a solid form, a paste form, or a gel form. More specifically, for example, a form in which the shape of the raw material remains as it is, such as soybean natto, a form in which the raw material is cleaved, shattered, or ground, a form in which these are dried, and the like can be used. Since the fermented food of the present invention has good texture and sweetness, it is suitable for eating as it is after fermentation.

The fermented food of the present invention can also be processed and used. The type of food to which the present invention is directed is not particularly limited, and any form may be used as long as it includes the fermented food of the present invention. Since the fermented food of the present invention has good texture and sweetness, it is preferable to use the fermented food for, for example, seasoning, sweets, baked confectionery, steamed confectionery, frozen confectionery, chocolate confectionery, gum, candy, gummy, cream, jam, a fermented food, a processed food, a cooked food, a retort food, or a cereal food, a solid food for vegetable intake, or a dosage form food (health and nutrition (supplement) food). In addition, the food includes food cooked so as to be ready to eat at home. More specifically, the examples thereof include a form in which the fermented food is fired or boiled, a form in which the fermented food is shattered or ground, a dried form of these, a dried powder form of these, and the like. Furthermore, the fermented food of the present invention may be eaten in a form of being mixed with other food products (including fermented foods and unfermented foods), seasoning, and the like, or may be subjected to processing and cooking. This makes it possible to impart appropriate sweetness and texture to the cooked food. In addition, sterilization or low-temperature distribution may be performed in order to suppress over-fermentation due to koji or Bacillus subtilis natto.

### 2. Method for Producing Fermented Food

The present invention, in one aspect thereof, relates to a method for producing a fermented food by adding a powder having an α-amylase activity to a solid raw material, the method satisfying all of the following stages (I) to (III): (I) a stage of adjusting a composition satisfying the following conditions: (a) the raw material is one or more kinds of edible plants selected from the group consisting of beans, cereals, vegetables, and nuts and seeds; and (b) a salt content is 1000 mg or less per 100 g of the fermented food, (II) a stage of adding a powder having α-amylase activity and/or Bacillus subtilis natto to the composition of (I), and (II) a stage of fermenting the composition of (II) (In the present specification, the “production method of the present invention” may be used.). This is described below.

With respect to the matters described in “1. fermented food” above, the description thereof is also incorporated in this section.

The production method of the present invention is not greatly different from a general method for producing itohiki-natto (sticky natto) using soybeans as a raw material, except that fermentation is performed using the raw material in the presence of koji.

A general method for producing itohiki-natto using soybeans as a raw material basically includes (1) preparation of steamed soybeans or boiled soybeans by immersion of raw soybeans and heating in a liquid (water vapor), (2) inoculating, (3) fermenting, and (4) aging, and thus the details of the method for producing a fermented food is described below according to this.

### (1) Preparation of Steamed Raw Material or Boiled Raw Material by Immersion of Raw Material and Heating in Liquid (Stage I)

First, in order to obtain a steamed raw material or a boiled raw material, the raw material is immersed and heated in a liquid.

The raw material can be used as it is, but it is common to use a raw material subjected to a drying treatment (dried product). It is also possible to use a raw material subjected to mechanical processing such as pulverization or cleavage, or chemical processing such as drying treatment or solution treatment. In the case of using a solid raw material, it is preferable to adjust the raw material before being subjected to the water absorption treatment to a diameter or major axis length of 3 to 50 mm.

In the production method of the present invention, in the same manner as general soybean natto, the raw material is immersed and heated in a liquid by a conventional method to be used as a steamed raw material or a boiled raw material.

That is, in the production method of the present invention, heating in a liquid is performed in order to use a raw material as a steamed raw material or a boiled raw material by a conventional method. Steaming is suitable in terms of preventing run-off of the components.

It is desirable to immerse the raw materials in water and swell the raw materials before steaming or boiling.

As a specific preparation procedure of the steamed raw material, for example, a method of immersing the raw material in water at 4° C. for about 6 to 24 hours, draining the water, and steaming with steam at 100 to 135° C. for 5 to 30 minutes can be adopted. At this time, for example, a method of steaming under pressure under a high pressure condition of 0.10 to 0.22 MPa can also be adopted. The steaming step is not performed at a time, and the step can be divided into a plurality of steps by once depressurizing and then pressurizing again. Further, water addition may be performed after each steaming step. When the water temperature at the time of immersion is high, bacterial contamination may occur, and therefore it is preferable to perform immersion at 4° C.

As a specific preparation procedure of the boiled raw material, for example, a method of immersing the raw material in water at 4° C. for about 6 to 24 hours, and then boiling the raw material in hot water at 90 to 100° C. for 20 to 50 minutes can be adopted.

(2) Mixing of Powder Having α-Amylase Activity, Inoculation of Bacillus subtilis Natto (Stage II)

A powder having α-amylase activity is mixed with the steamed raw material or the boiled raw material thus obtained. In the case of inoculating Bacillus subtilis natto, a powder having α-amylase activity is added and mixed before or during fermentation with Bacillus subtilis natto, and the raw material is fermented with Bacillus subtilis natto in the presence of the powder having α-amylase activity. In the case of inoculating Bacillus subtilis natto, for example, when an inoculation step of inoculating Bacillus subtilis natto is performed, a step of mixing koji with a powder having α-amylase activity may be performed, or a mixing step of inoculating Bacillus subtilis natto into a raw material and then mixing the raw material with a powder having α-amylase activity may be performed.

In the production method of the present invention, the raw material and the powder having α-amylase activity are mixed in a prodetermined amount. Mixing of the raw material and the powder having α-amylase activity may be performed at any time point before or after inoculation of Bacillus subtilis natto as long as it is before the start of the fermentation step described later. Therefore, fermentation may be performed by inoculating Bacillus subtilis natto into a mixture of the powder having α-amylase activity and the raw material, or fermentation may be performed after mixing a product obtained by inoculating Bacillus subtilis natto into the raw material and the powder having α-amylase activity.

As the powder having α-amylase activity, it is preferable to use a koji powder. As the koji used at this time, the koji-made koji is particularly preferable, and the definition of koji, the raw materials used, molds and the like, and preferred ones thereof are included as described in detail in the above “1. fermented food”.

With respect to the ratio of the powder having α-amylase activity to the raw material, as shown in the above “1. fermented food”, the mass ratio of the powder having α-amylase activity to the raw material is 1:3 or more and 1:10 or less, and the preferable mass ratio and the reason thereof are as described in detail in the above “1. fermented food”.

The koji powder of the present invention preferably has a step of retaining at 40° C. or less for 1 hour or more before being used in the stage (II). The heat-retaining temperature is preferably 4 to 40° C. or less, specifically 40° C. or less, preferably 35° C. or less, more preferably 30° C. or less, and further preferably 25° C. or less, and the lower limit thereof is not particularly limited, and is 4° C. or more, preferably 10° C. or more, more preferably 15° C. or more, and further preferably 20° C. or more for industrial reasons. The heat-retaining time is preferably 1 hour to 700 hours, specifically 700 hours or less, or 600 hours or less, or 500 hours or less, or 400 hours or less, or 300 hours or less, preferably 240 hours or less, and further preferably 168 hours or less. The lower limit thereof is not particularly limited, and is, for example, 1 hour or more, preferably 3 hours or more, and more preferably 4 hours or more. By using the heat-retained A koji V powder under the above conditions, a dry mealiness mouthfeel of the fermented food can be suppressed. In addition, the growth of various bacteria such as yeast can be suppressed, and the quality of the fermented food can be stabilized.

Next, Bacillus subtilis natto to be inoculated is described.

In the production method of the present invention, the state of Bacillus subtilis natto added as a Bacillus subtilis natto starter is not particularly limited, and it is preferable to use a spore state capable of directly inoculating a high-temperature raw material immediately after steaming in order to prevent bacterial contamination.

Any Bacillus subtilis natto can be employed as the Bacillus subtilis natto used in the Bacillus subtilis natto starter. For example, common commercially available bacteria such as Miyagino bacteria (trade name: pure culture Bacillus subtilis natto (Miyagino Bacillus subtilis natto) (manufactured by Miyagino Manufacturing Co., Ltd.), Takahashi bacteria (trade name: NATTOMOTO) (manufactured by Yuzo Takahashi Laboratory), and Naruse bacteria (trade name: powder Bacillus subtilis natto) (manufactured by Naruse Fermentation Chemical Laboratory) can be used, but various kinds of bacteria strains such as mutant strains and genetically modified strains having specific properties can also be used.

Incidentally, Bacillus subtilis natto is a bacterium that is classified as Bacillus subtilis, and is classified Bacillus subtilis var. natto or Bacillus subtilis (natto) as a variant of Bacillus subtilis, distinct from Bacillus subtilis, or as Bacillus natto which is a closely related species of Bacillus subtilis.

In order to ensure uniform fermentation, when inoculating the Bacillus subtilis natto starter into a steamed raw material or a boiled raw material, it is desirable to add the starter by seeding or spraying, and then perform mixing or the like so that the Bacillus subtilis natto becomes uniform with the steamed or boiled raw materials. Preferably, it is preferable to prepare a spore suspension of the Bacillus subtilis natto, add and use the spore suspension in a liquid state.

Here, as the spore suspension, a culture solution obtained by culturing the Bacillus subtilis natto in a liquid medium having a component suitable for spore formation can be used.

The component of the liquid medium is not particularly limited as long as it is a liquid medium containing a medium component such as a carbon source, a nitrogen source, and inorganic salts, which enables spore formation and growth of Bacillus subtilis natto and is usually used for culture of Bacillus subtilis natto, and may be a synthetic medium or a natural medium.

Among the medium components, examples of the carbon source include saccharides such as glucose, sucrose, galactose, mannose, starch, and starch decomposition products, organic acids such as citric acid, examples of the nitrogen source include peptone, meat extract, casein hydrolysate, ammonia, ammonium sulfate, and ammonium chloride, and examples of the inorganic salts include sodium chloride, potassium chloride, calcium chloride, sodium sulfate, sodium hydrogen sulfate, sodium nitrate, potassium phosphate, ferric chloride hexahydrate, magnesium sulfate heptahydrate, manganese chloride tetrahydrate, and ferrous sulfate.

The medium may contain yeast extract, malt extract, soybean flour, vitamins (biotin and the like), and the like. When a mutant strain of Bacillus subtilis natto requiring a specific nutrient component due to a genetic defect or the like is used, the medium composition may be changed as appropriate.

The number of Bacillus subtilis natto to inoculated is not particularly limited as long as it is a bacteria concentration according to a common method of soybean natto, but is usually 103 to 10° per 1 g of the steamed raw material or the boiled raw material. The product temperature of the raw material at the time of inoculating is not particularly limited, but it is preferable to inoculate it at a high temperature of about 55 to 95° C. in order to prevent bacterial contamination at the time of inoculating.

The raw material inoculated with the Bacillus subtilis natto is preferably filled in an individual container for one to several servings, and then subjected to fermentation described later in the individual container. As a traditional method, it is also possible to fill a boiled straw wrapper.

In addition, it is also possible to perform fermentation in a container having a volume of several liters or the like, but it is not desirable to use a large container in consideration of the fact that a temperature change is less likely to be transmitted to the beans in the central part when the volume value with respect to the surface area increases.

As the individual container, any container can be used as long as it can be filled with beans. Specifically, it is possible to use a container molded using a foamed sheet made of various synthetic resins such as a styrene-modified polyolefin-based resin generally used in natto, a polystyrene-based resin such as polystyrene, high-impact polystyrene, and a styrene-ethylene copolymer, a polyolefin-based resin such as polyethylene, polypropylene, and an ethylene-vinyl acetate copolymer, and a polyester-based resin such as polyethylene terephthalate, or a cup-shaped paper container.

In addition, in one aspect of the present invention, as the shape of the container, a shape that enables stirring (stirring) for eating directly using the container is preferable.

In addition, an aspect in which sealing by a lid or sealing can be performed after fermentation is suitable.

### (3) Fermentation (Stage II)

In the production method of the present invention, fermentation is performed after performing a mixing step of mixing koji with a steamed raw material or a boiled raw material in this manner (an inoculation step of further inoculating Bacillus subtilis natto as necessary).

### Fermentation Conditions

In the fermentation of the production method of the present invention, it is essential to maintain the product temperature of the raw material at a substantially normal fermentation temperature zone for a predetermined time from the start of fermentation.

The normal fermentation temperature zone refers to a temperature zone in which the product temperature is 30 to 60° C. When the fermentation temperature is lower than a predetermined temperature, a good flavor of the fermented food is not imparted, and it becomes difficult to keep the specific component within a predetermined range. When the fermentation temperature is higher than a predetermined temperature, ammonia, lower fatty acid, and the like are excessively generated, which is not preferable.

The phrase “maintain the product temperature of the raw material at a substantially normal fermentation temperature zone for a predetermined period of time from the start of fermentation.” does not mean that the temperature does not completely deviate from the temperature zone. For example, when the temperature is in a slight temperature range (for example, within 3° C., preferably within 2° C.) and for a slight time (for example, within 20 minutes, preferably within 10 minutes), even when the product temperature deviates from the temperature zone, it means that the fermentation conditions are satisfied.

In the fermentation of the production method of the present invention, the predetermined time (fermentation time) for maintaining the fermentation temperature zone may be 5 to 23 hours. The lower limit of the fermentation time may be 5 hours or more, preferably 6 hours or more, further preferably 7 hours or more. In the fermentation, when the fermentation time is shorter than a predetermined time, the fermentation does not sufficiently proceed, and the specific component easily deviates from a predetermined range.

The upper limit of the fermentation time may be 23 hours or less, preferably 22 hours or less, further preferably 21 hours or less. If the fermentation time is longer than a predetermined time, the fermentation proceeds too much, and the content of lower fatty acid particularly increases, which is not preferable.

Therefore, as fermentation conditions, fermentation is preferably performed at a product temperature in the range of 30° C. or more and 60° C. or less for 5 hours or more and 23 hours or less.

In the fermented food of the present invention, in order to adjust the specific components to a prodetermined range, high-temperature fermentation is preferably performed by maintaining the fermented food at a higher temperature zone (product temperature is 45° C. or more and 60° C. or less) for 4 to 22 hours in a normal fermentation temperature zone, because the enzyme activity derived from koji functions efficiently and the specific components are easily contained within a predetermined range. Specifically, Bacillus subtilis natto is inoculated into a steamed raw material or a boiled raw material, and high-temperature fermentation is performed by maintaining the product temperature at 45° C. or more and 60° C. or less for 4 hours or more and 22 hours or less.

The lower limit of the fermentation temperature in the case of high-temperature fermentation can be 45° C. or more, preferably 46° C. or more, and more preferably 47° C. or more. The upper limit of the fermentation temperature in the case of high-temperature fermentation can be 54° C. or less, preferably 53.75° C. or less, and more preferably 53.5° C. or less. Therefore, the fermentation temperature in the case of high-temperature fermentation is 45° C. or more and 54° C. or less, preferably 46° C. or more and 53.75° C. or less, and more preferably 47° C. or more and 53.5° C. or less.

The lower limit of the high-temperature fermentation time is preferably maintained for 4 hours or more, preferably 5 hours or more, more preferably 8 hours or more, and further preferably 10 hours or more. The upper limit of the high-temperature fermentation time is preferably maintained at 22 hours or less, preferably 20 hours or less, more preferably 18 hours or less, and further preferably 16 hours or less. Therefore, the fermentation time in the case of high-temperature fermentation is 4 hours or more and 22 hours or less, preferably 5 hours or more and 20 hours or less, more preferably 8 hours or more and 18 hours or less, and further preferably 10 hours or more and 16 hours or less.

In a preferred aspect of the present invention, it is preferable to further perform short-time fermentation (fermentation step 2) at a high temperature after the fermentation step (fermentation step 1) described above (stage IV). The fermentation temperature in fermentation step 2 is, for example, 55° C. or more and 62° C. or less, more preferably 57° C. or more and 61° C. or less, and further preferably 59° C. or more and 60.5° C. or less. The fermentation time in the fermentation step 2 is, for example, 0.5 hours or more and 3 hours or less, preferably 1 hour or more and 2 hours or less.

In the production method of the present invention, the characteristic specific component is produced in the fermentation step under the condition that the secondary fermentation is sufficiently suppressed in the aging step described later or the storage and distribution of the fermentation product. Therefore, during the fermentation step, the content of the specific component contained in the fermented food can be appropriately monitored by the measurement method described later, and the fermentation step can be stopped when the content reaches a predetermined content. In addition, when the amount of the specific component is insufficient until the end of fermentation, the preparation may be performed by adding the insufficient specific component so as to fall within a predetermined range.

In order to accurately adjust the temperature conditions in this fermentation step, it is preferable to use a fermentation chamber or the like with heating and cooling functions.

### (4) Aging (Stage V)

After completion of the fermentation step, in order to suppress quality deterioration such as production of ammonia by secondary fermentation, aging is usually performed at a low temperature of 3° C. or more and less than 10° C., preferably 3° C. or more and less than 8° C., more preferably 3° C. or more and less than 6° C., for 6 hours to 3 days, preferably 8 hours to 2 days, more preferably about 24 hours, so that production of a fermented food is completed.

### 3. Method for Imparting Sweetness to Fermented Food

The present invention, in one aspect thereof, relates to a method for imparting a sweetness to a fermented food, in which when a sweetness is imparted to a fermented food using beans, cereals, vegetables, and nuts and seeds as raw materials, koji is used, and a salt content is 1000 mg or less per 100 g of the fermented food (In the present specification, it may be referred to as “the sweetening method of the present invention”). This is described below.

With respect to the matters described in the above “1. fermented food” and the above “2. Method for producing fermented food”, the description thereof is also incorporated in this section.

In the imparting method of the present invention, it is not possible to impart a sweetness to the fermented food in a case where the koji is not used. In addition, even when koji is used, sweetness cannot be imparted to the fermented food unless the salt content is 1000 mg or less per 100 g of the fermented food.

In a preferred aspect of the present invention, umami taste and richness can be imparted to a fermented food in addition to sweetness.

In a further preferred aspect of the imparting method of the present invention, a chestnut like flavor can be imparted to the fermented food in addition to sweetness.

### 4. Method for Imparting Al Dente-Like Texture to Fermented Food

The present invention, in one aspect thereof, relates to a method for imparting an al dente-like texture to a fermented food using koji when imparting the al dente-like texture to a fermented food containing beans, cereals, vegetables, and nuts and seeds as raw materials (In the present specification, it may be referred to as “method for imparting a texture of the present invention”.). This is described below.

With respect to the matters described in the above “1. fermented food” and the above “2. Method for producing fermented food”, the description thereof is also incorporated in this section.

In the method for imparting a texture of the present invention, it is not possible to impart an al dente-like texture to a fermented food in a case where koji is not used.

In a preferred aspect of the imparting method of the present invention, a texture like a chestnut can also be imparted to the fermented food.

### EXAMPLES

Hereinafter, the present invention is described in detail based on Examples, but the present invention is not limited by these Examples.

### Test Methods

Various measurement and evaluation methods in Test Example 1 and Test Example 2 below are shown below.

### <Measurement of Respective Starch Contents of Raw Material and Koji>

The starch content (% by mass) in koji powder was measured according to AOAC method 996.11 (AOAC, 2005) in accordance with the method in STANDARD TABLES OF FOOD COMPOSITION IN JAPAN—2020—(Eighth Revised Edition). The starch content (% by mass) of the raw material was calculated with reference to the item “Starch” described in STANDARD TABLES OF FOOD COMPOSITION IN JAPAN—2020—(Eighth Revised Edition).

### <Measurement of Dietary Fiber Content of Raw Material>

The dietary fiber content (% by mass) of the raw materials before processing was calculated with reference to the item of “Total amount of dietary fiber” described in STANDARD TABLES OF FOOD COMPOSITION IN JAPAN—2020—(Eighth Revised Edition).

### <Pretreatment of Salt Analysis of Fermented Food Sample>

As a pretreatment for sodium analysis for determination of the salt content, acidolysis was performed in the following procedure. That is, 10 ml of nitric acid was added to 0.5 g of a sample, and then a heat treatment was performed using an acidolysis device DigiPREP (manufactured by GL Sciences Inc.) under the following conditions.

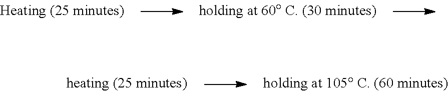

After the sample was cooled to 60° C., 2 ml of hydrogen peroxide water was added, and the following heat treatment was further performed.

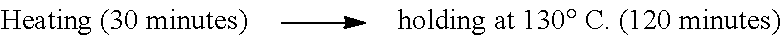

The acidolysis solution subjected to the above treatment was diluted with 1% nitric acid so as to be 5000 times the amount of the sample, and then subjected to sodium analysis by ICP-MS 7000 (manufactured by Agilent Technologies). The measured sodium content was converted to the equivalent of salt (2.54 times) to calculate the salt content.

### <Measurement of Viable Cell Count of Fermented Food Sample>

The viable cell count in the sample was measured by culturing a diluted solution obtained by diluting the sample suspension on an agar medium to measure the number of colonies.

That is, the sample was put into a bag with an attached filter attached to a paddle type blender “Stomacher”, a phosphate buffer solution was injected, and the bag was shaken with the above Stomacher. Subsequently, the mixture was diluted with a phosphate buffer solution, then mixed with a standard agar medium (manufactured by atect Corp.), cultured at 37° C. for 18 hours, and the number of colonies appearing therein was calculated.

In addition, in Reference Examples b1, b3, and b4 in which Bacillus subtilis natto was not inoculated, the number of appearance colonies was significantly small, and the appearance of appearance colonies was not a bacillus such as Bacillus subtilis natto but a shape derived from yeast or mold, and therefore was not detected (ND).

### <Pretreatment of Sample for Measuring Sugar Composition and Amino Acid Composition of Fermented Food Sample>

Pretreatment of the sample for measuring the sugar composition and amino acid composition was performed by the following method.

10 g of a sample was placed in a synthetic resin pouch and then pressed with a metal spatula to form a paste. Next, 50% ethanol was added little by little, and when it became a thick liquid, it was transferred to a 100 ml volumetric flask. All samples were placed in a volumetric flask with rinsing and filled up with 50% ethanol.

The obtained sample suspension was subjected to sonication for 20 minutes, then subjected to centrifugal treatment (10,000×g, 5 minutes), and then the supernatant part was filtered with filter paper (No. 2) and collected as a pretreatment liquid.

### <Sugar Composition Analysis of Fermented Food Sample>

Regarding the analysis of various saccharides, a filtrate obtained by filtering a diluted solution obtained by diluting the pretreatment liquid subjected to the above pretreatment with water so that the content of saccharides contained at the highest concentration was about 1% by mass or less through a 0.45 μm filter was subjected to HPLC measurement.

As an HPLC system, Prominence (manufactured by SHIMADZU CORPORATION) was used. As a measurement column, HILICpak VG-50 4E HPLC was used, 80% acetonitrile was used as a mobile phase, and detection was performed with an indicating refractometer (Shodex RI-201H).

As the standard, one obtained by diluting each standard of glucose (manufactured by Sigma-Aldrich Co. LLC.) and maltose (manufactured by Sigma-Aldrich Co. LLC.) with water was used. The obtained measured value was converted according to the dilution rate of the sample to calculate the measured value.

### <Amino Acid Composition Analysis of Fermented Food Sample>

In the analysis of amino acids, free amino acids were measured in a filtrate obtained by filtering a diluted solution obtained by diluting the pretreated solution subjected to the above pretreatment liquid to about 2 mg/100 ml with a lithium citrate solution (pH 2.2) through a 0.45 μm filter.

For the measurement, an amino acid analyzer JLC-500/V2 (manufactured by JEOL Ltd.) was used.

As a standard, an amino acid mixture standard solution B type for automatic amino acid analysis (manufactured by FUJIFILM Wako Pure Chemical Corporation) diluted to 2 mg/100 ml was used.

### <Analysis of Branched Chain Fatty Acid in Fermented Food Sample>

The branched chain fatty acid content of the fermented food sample was measured as follows.

The fermented food sample was made into a paste by a blender, 4 ml of water was added to 1 g of the paste-like sample, and the mixture was ground in a mortar. The mortar was capped with an aluminum foil or the like and incubated at 4° C. for 60 minutes. The suspension was transferred to the full 1.5 ml tube and centrifuged for 10 minutes at 20,000×g, 5° C. 0.75 ml of the supernatant was transferred to a new tube and 0.75 ml of 0.8% phosphoric acid was added and mixed. Centrifugation was performed at 20,000×g and 5° C. for 10 minutes, the supernatant was filtered through a 0.45 μm filter, and the filtrate was subjected to HPLC measurement.

As an HPLC system, an e2695 separation module (manufactured by Waters Corporation) was used. Detection was performed with an electrical conductivity detector (Polarity: +, Responese: STD, Gain: 1 μS/cm, temperature 53° C.) using a guard column of hodex KC-810P (6 mmφ×50 mm), a measurement column of hodex KC-811 (8 mmφ×300 mm) (manufactured by Showa Denko K.K.), a 4 mM p-toluenesulfonic acid aqueous solution as a mobile phase, and a 16 mM Bis-Tris aqueous solution containing 4 mM p-toluenesulfonic acid and 80 μM EDTA as a reaction liquid.

### Standard;

In 100 ml of water, 0.1053 g of acetic acid, 0.0991 g of propionic acid, 0.1014 g of isobutyric acid, 0.1024 g of butyric acid, 0.0991 g of isovaleric acid, and 0.1014 g of valeric acid were dissolved, and the solution was diluted 10 times and used.

### <Analysis of 2-Phenylethyl Alcohol, 4-Vinyl-2-Methoxyphenol in Fermented Food Sample>

Analysis of 2-phenylethyl alcohol and 4-vinyl-2-methoxyphenol was performed by injecting an aromatic component in each of the samples of Examples and Reference Examples into GC-MS by a dynamic headspace method.

As a measuring instrument, a one-dimensional and two-dimensional switching GC-MS (GC section: LTM series II connected to HP7890 Series GC System (both manufactured by Agilent Technologies), injection port: TDU2/CIS4 (manufactured by GERSTEL K. K.), autosampler: MPS (manufactured by GERSTEL K. K.)) manufactured by GERSTEL K. K. was used.

Injecting the sample into the instrument was performed by a dynamic headspace method. Specifically, 0.27 g of each specimen was weighed into a 10 mL flat-bottom vial and then sealed, and a sample volatilized by 60 mL nitrogen gas purge was adsorbed with an adsorption resin (Tenax column) corresponding to the properties of the analytical component, and then injection treatment was performed under the following conditions using a thermal desorption system.

### [GC-MS Condition (Dynamic Head Space (DHS) Injection Method)]

Apparatus: 7890b (GC) and 5977b (MS) manufactured by Agilent Technologies, Inc., auto sampler
MPS (Multipurpose Sampler) manufactured GERSTEL K. K.
Adsorption resin: Tenax
Incubation temperature: 80° C.
Nitrogen gas purge amount: 60 mL
Nitrogen gas purge flow rate: 10 ml/min

As the capillary column, DB-WAX (length: 30 m, inner diameter: 250 μm, film thickness: 0.25 μm, for LTM) (manufactured by Agilent Technologies) was used as a one-dimensional column. Helium was used as the carrier gas.

Sample injection conditions were as follows.
CIS4:
   Holding at 10° C. for 0.5 minutes, and then heating to 240° C. at 720° C./min.
TDU2:
   Holding at 30° C. for 0.2 minutes, and then heating to 240° C. at 240° C./min.

In the measurement, after injection was performed under the above injection conditions, separation was performed with a one-dimensional column, and a selected ion detection (SIM) mode was performed. The column oven conditions of DB-WAX (one-dimensional column) were as follows.
DB-WAX (one-dimensional column):

Holding at 40° C. for 3 minutes, then raising the temperature to 240° C. at 5° C./min, and holding for 7 minutes.

For each measurement specimen, the concentration of the measurement specimen was calculated by an absolute calibration curve method from the area of a quantitative ion (see the following) when a solution obtained by diluting each standard substance of 2-phenylethyl alcohol and 4-vinyl-2-methoxyphenol with water was measured in the same manner as the specimen in a selected ion detection (SIM) mode. As the standard substance, the same standard substance as that used for addition to the Example sample was used.

### (Quantitative Ion)

2-phenylethyl alcohol Quantitative ion: 91, confirmation ion 1: 122, confirmation ion 2: 92
4-vinyl-2-methoxyphenol Quantitative ion: 135, confirmation ion 1: 150, confirmation ion 2: 107

### Test Example 1: Fermentation Test Using Beans, Cereals, Vegetables, and Nuts and Seeds as Raw Materials

### Examples A1 to A37; Production of Samples

The samples of Examples A1 to A37 were produced by the following procedure. (However, among the above, Example A9 is a fermented food fermented only with koji, since Bacillus subtilis natto was not inoculated. As raw materials, beans, cereals, vegetables, and nuts and seeds shown in Tables 2, 4, and 6 were used.

### (Preparation of Steamed Raw Material)

Steamed raw materials used in Examples were prepared by the following method.

The beans and cereals were used as they were without adjusting the size of the raw materials. Chickpeas and yellow peas subjected to crushing treatment were also used. The vegetables were adjusted to a size of 50 mm square or less using a kitchen knife or the like. The diameter or major axis length of each raw material before water absorption treatment is shown in Tables 3,5, and 7. The raw material was lightly washed with water, and treated in a refrigerator at 4° C. for 18 hours in a state of being immersed in water, so that the water was sufficiently absorbed by the raw material, and then the water was drained. Subsequently, the immersed raw material from which water had been removed was subjected to a steaming step. Specifically, the immersed raw materials were put in a metal container, placed in a steaming pot (steaming pot for test manufactured by HARADA SANGYO CO., LTD.), heated to a temperature of 98° C., then pressurized under the conditions shown in Table 1 below, and then depressurized to perform steaming. The “pressure” in Table 1 refers to a pressure applied to atmospheric pressure.

TABLE 1

| Lapse time (minutes) | Holding time (minutes) | Pressure (kPa) |
|---|---|---|
| 0 | 0 | 0 |
| 5 | 5 | 100 |
| 7 | 2 | 130 |
| 9 | 2 | 160 |
| 23 | 14 | 100 |
| 26 | 3 | 50 |
| 28 | 2 | 0 |

In Example A9, only koji was mixed with the steamed raw material thus obtained by the method described later. In another example, koji was mixed with the steamed raw material immediately after steaming by the method described later to inoculate Bacillus subtilis natto.

### (Fermentation of Nato)

Each of the steamed raw materials after completion of steaming in the above procedure was inoculated in the following procedure. As the Bacillus subtilis natto, Bacillus subtilis K-2 strain (NITE BP-1577), Bacillus subtilis K2-7 strain (NITE BP-03083), Bacillus subtilis B-1 strain (NITE BP-08584), and Bacillus subtilis MZ-21113 strain (NITE BP-02420) were used.

These Bacillus subtilis natto strains were inoculated into 10 ml/test tube of a spore formation medium (YE) shown in the following Table 2, and shake-cultured at 37° C. for 200×g for 24 hours to obtain a spore suspension.

TABLE 2
Spore formation medium (YE)

| yeast extract* | 20.0 g/L |
|---|---|
| NaCl | 5.0 g/L |
| pH (adjusted with NaOH) | 7.1 |

*(Meast (trademark) P2G: manufactured by ASAHI FOOD & HEALTHCARE, LTD)

As the Bacillus subtilis natto strain used in Examples, the above-mentioned Bacillus subtilis natto strains were used, and a spore suspension cultured in a liquid medium was diluted with water so that about 1000 Bacillus subtilis natto were present per 1 g of boiled beans, and the strains were inoculated into a steamed raw material.

Immediately after inoculation of Bacillus subtilis natto, koji was mixed at a ratio described in Tables 3, 5, and 7 described later. A yellow koji powder (koji mold: Aspergillus oryzae, manufactured by Hishiroku Co., Ltd.), which was rice koji, was used as koji, and the materials were mixed at the proportions shown in Tables 3, 5, and 7. The steamed raw materials after the mixing of the koji were placed in a polystyrene container to perform fermentation. The koji was in the form of a powder, the dry basis moisture content thereof was 15% by mass or less, and the d50 after sonication was 500 μm or less. In addition, the coverage of the koji with respect to the surface area of the raw material ((the surface area of the edible plant serving as the raw material to which the powder having an α-amylase activity is attached/the surface area of the edible plant serving as the raw material)×100) was 50% or more.

However, in Example A9, without inoculating Bacillus subtilis natto, only the koji was mixed with the steamed raw material and dispensed into a polystyrene container.

The control temperature (hereinafter, abbreviated as room temperature) of the chamber during fermentation of the sample is as shown in Tables 3, 5, and 7. As a fermentation chamber, a natto fermentation chamber (fermentation chamber for test manufactured by HARADA SANGYO CO., LTD.) was used. The fermentation was performed at room temperature of 32 to 55° C. for 8 to 14 hours to perform main fermentation. Thereafter, the room temperature was raised to 60° C., and a high temperature treatment was performed for 1.5 hours. Example A8 was treated at room temperature of 37° C. for 1.5 hours without being subjected to a high temperature treatment.

The sample after completion of the fermentation step was aged by cooling in a refrigerator at 4° C. The quality evaluation was performed on each sample after aging. The results of the samples subjected to the sensory evaluation are shown in Table 3 and Table 5. Analysis results of each component are shown in Tables 4, 6, and 8. In the table, the part not measured was regarded as “-”.

### <Sensory Evaluation>

Sensory evaluation was performed by the following method.

Evaluation of each sample was performed under the following conditions. Each evaluation was performed by the following four trained sensory inspectors, and a value rounded off to the first decimal place of the average score was described as the score is shown in Table 4.

The sensory inspector conducted identification training of the following A) and B), and particularly selected a person with excellent results as a panel.
A) A taste discrimination test in which for each of the five tastes (sweetness: taste of sugar, sourness: taste of tartaric acid, umami: taste of sodium glutamate, saltiness: taste of sodium chloride, bitterness: taste of caffeine), an aqueous solution each with a concentration close to the threshold value of each component is prepared, and samples of each taste are accurately identified from a total of seven samples obtained by adding two distilled water thereto.
B) A concentration difference discrimination test that accurately discriminates a concentration difference between five types of saline solution and acetic acid solution having slightly different concentrations.

### (Sweetness)

Evaluation was performed with the following five grades of strength. Here, the term “harshness” refers to a taste in which scum is strong and a throat or tongue feels harsh, and a taste that is not preferred, mainly bitterness and astringency.
5: Sweetness is felt, and harshness of a raw material is not felt.
4: Slight sweetness is felt, and harshness of a raw material is not felt.
3: A certain degree of sweetness is felt, and harshness of a raw material is not felt.
2: Sweetness is slightly felt, and harshness of a raw material is slightly felt.
1: No sweetness is felt, and harshness of a raw material is felt.

### (Natto Odor)

Examples of components causing a natto odor include lower branched fatty acids such as isovaleric acid and isoentangled acid; pyrazines such as 2,5 dimethylpyrazine, trimethylpyrazine, and tetramethylpyrazine. Four skilled panels were used, and the natto odor was evaluated in the following five grades.
5 It is very preferable because the natto odor is not felt.
4 It is preferable because the natto odor is hardly felt.
3 It is somewhat preferable because the natto odor is slightly felt.
2 It is not preferable because the natto odor is felt.
1 It is not very preferable because the natto odor is strongly felt.

### (Texture)

The al dente-like texture refers to a unique texture generated by a synergistic effect of the hardness of the central part and the gradient of hardness from the vicinity of the soft surface to the central part having hardness.
5: It is very preferable because the al dente-like texture is very felt.
4: It is preferable because the al dente-like texture is felt.
3: It is somewhat preferable because the al dente-like texture is slightly felt.
2: It is not preferable because the al dente-like texture is slightly weak felt.
1: It is not preferable because the al dente-like texture is weak felt.

TABLE 3

| | Raw materials | | | | | | | | Bacillus subtilis | | | | |
|---|---|---|---|---|---|---|---|---|---|---|---|---|---|
| | | | Dietary | Diameter | Rice koji | | | | natto | | Main | | |
| | | Starch | fiber | or major | | Starch | | | | Number | fermentation | | 60° C. |
| | | content | content | axis | | content | ratio of | Salt | | of inocu- | Temper- | | step |
| | | (% by | (% by | length | | (% by | koji:raw | content | Bacteria | lation | ature | Time | Time |
| | Type | mass) | mass) | (mm) | Type | mass) | materials | mg/100 g | strain | (cells/g) | (° C.) | (h) | (h) |
| Example | Yellow | 40.8 | 17.4 | 11 | Yellow koji | 55.2 | 1:6 | 0 | B-1 | 1000 | 50 | 11 | 1.5 |
| A1 | pea | | | | powder | | | | | | | | |
| Example | Yellow | 40.8 | 17.4 | 11 | Yellow koji | 55.2 | 1:6 | 0 | B-1 | 1000 | 52 | 8 | 1.5 |
| A2 | pea | | | | powder | | | | | | | | |
| Example | Yellow | 40.8 | 17.4 | 11 | Yellow koji | 55.2 | 1:6 | 0 | B-1 | 1000 | 55 | 8 | 1.5 |
| A3 | pea | | | | powder | | | | | | | | |
| Example | Yellow | 40.8 | 17.4 | 11 | Yellow koji | 55.2 | 1:6 | 0 | B-1 | 1000 | 55 | 11 | 1.5 |
| A4 | pea | | | | powder | | | | | | | | |
| Example | Yellow | 40.8 | 17.4 | 11 | Yellow koji | 55.2 | 1:6 | 0 | None | 0 | 55 | 11 | 1.5 |
| A5 | pea | | | | powder | | | | | | | | |
| Example | Yellow | 40.8 | 17.4 | 11 | Yellow koji | 55.2 | 1:8 | 0 | B-1 | 1000 | 52 | 8 | 1.5 |
| A6 | pea | | | | powder | | | | | | | | |
| Example | Yellow | 40.8 | 17.4 | 11 | Yellow koji | 55.2 | 1:10 | 0 | B-1 | 1000 | 52 | 8 | 1.5 |
| A7 | pea | | | | powder | | | | | | | | |
| Example | Yellow | 40.8 | 17.4 | 11 | Yellow koji | 55.2 | 1:6 | 0 | B-1 | 1000 | 32 | 13 | 37° C., |
| A8 | pea | | | | powder | | | | | | | | 5 h |
| Example | Yellow | 40.8 | 17.4 | 11 | Yellow koji | 55.2 | 1:6 | 0 | None | 0 | 55 | 11 | 1.5 |
| A9 | pea | | | | powder | | | | | | | | |
| Example | Yellow | 40.8 | 17.4 | 11 | Yellow koji | 55.2 | 1:6 | 0 | K2-7 | 1000 | 52 | 8 | 1.5 |
| A10 | pea | | | | powder | | | | | | | | |
| Example | Yellow | 40.8 | 17.4 | 11 | Yellow koji | 55.2 | 1:6 | 0 | MZ- | 1000 | 55 | 8 | 1.5 |
| A11 | pea | | | | powder | | | | 21113 | | | | |
| Example | Yellow | 40.8 | 17.4 | 11 | Yellow koji | 55.2 | 1:6 | 0 | MZ- | 1000 | 55 | 11 | 1.5 |
| A12 | pea | | | | powder | | | | 21113 | | | | |

TABLE 4

| | Sugar content | | Branched chain | Free arginine |
|---|---|---|---|---|
| | (% by mass) | | fatty acid | content |
| | Glucose | Maltose | mg/100 g | mg/100 g |
| Example A1 | 14.82 | 1.32 | 3.5 | 15.6 |
| Example A2 | 13.82 | 1.83 | 2.1 | 10.8 |
| Example A3 | 15.05 | 1.56 | 1.3 | 8.7 |
| Example A4 | 16.07 | 1.39 | 1 | 12.4 |
| Example A5 | 11.24 | 1.96 | 0 | 24.3 |
| Example A6 | 13.14 | 2.04 | 2.6 | 3.0 |
| Example A7 | 9.74 | 1.51 | 3.2 | 2.0 |
| Example A8 | 9.26 | 1.67 | 6.1 | 8.5 |
| Example A9 | 15.59 | 2.13 | 0 | 19.6 |
| Example A10 | 12.65 | 1.72 | 2.4 | 7.1 |
| Example A11 | 12.56 | 1.6 | 0 | 12.4 |
| Example A12 | 16.36 | 1.73 | 0 | 14.3 |

TABLE 5

| | Raw materials | | | | | | Ratio | | | |
|---|---|---|---|---|---|---|---|---|---|---|
| | | | Dietary | Diameter | Rice koji | | of koji | | Bacillus subtilis | |
| | | Starch | fiber | or major | | Starch | and raw | | natto | |
| | | content | content | axis | | content | material | Salt | | Number of |
| | | (% by | (% by | length | | (% by | % by | content | Bacteria | inoculation |
| | Type | mass) | mass) | (mm) | Type | mass) | mass | mg/100 g | strain | (cells/g) |
| Example | Chickpea | 35.4 | 21.5 | 4 | Yellow | 55.2 | 1:6 | 0 | B-1 | 1000 |
| A13 | | | | | koji | | | | | |
| | | | | | powder | | | | | |
| Example | Chickpea | 35.4 | 21.5 | 4 | Yellow | 55.2 | 1:6 | 0 | B-1 | 1000 |
| A14 | | | | | koji | | | | | |
| | | | | | powder | | | | | |
| Example | Chickpea | 35.4 | 21.5 | 4 | Yellow | 55.2 | 1:6 | 0 | B-1 | 1000 |
| A15 | | | | | koji | | | | | |
| | | | | | powder | | | | | |
| Example | Chickpea | 35.4 | 21.5 | 4 | Yellow | 55.2 | 1:6 | 0 | B-1 | 1000 |
| A16 | | | | | koji | | | | | |
| | | | | | powder | | | | | |
| Example | Chickpea | 35.4 | 21.5 | 4 | Yellow | 55.2 | 1:6 | 0 | B-1 | 1000 |
| A17 | | | | | koji | | | | | |
| | | | | | powder | | | | | |
| Example | Chickpea | 35.4 | 21.5 | 4 | Yellow | 55.2 | 1:6 | 0 | B-1 | 1000 |
| A18 | | | | | koji | | | | | |
| | | | | | powder | | | | | |

| | | | | Viable cell | | | |
|---|---|---|---|---|---|---|---|
| | | | | count of | | | |
| | | | | Bacillus | | | |
| | Main | | 60° C. | subtilis | | | |
| | fermentation | | step | natto | Sensory | | |
| | Temperature | Time | Time | (1000 | | Natto | |
| | (° C.) | (h) | (h) | cells/g) | Sweetness | odor | Texture |
| Example | 50 | 8 | 1.5 | 620,000 | 3 | 3 | 4 |
| A13 | | | | | | | |
| Example | 50 | 11 | 1.5 | — | 4 | 4 | 5 |
| A14 | | | | | | | |
| Example | 52 | 8 | 1.5 | 99,000 | 4 | 3 | 5 |
| A15 | | | | | | | |
| Example | 52 | 11 | 1.5 | 120,000 | 4 | 4 | 5 |
| A16 | | | | | | | |
| Example | 55 | 8 | 1.5 | 700,000 | 3 | 5 | 4 |
| A17 | | | | | | | |
| Example | 55 | 11 | 1.5 | 1,000,000 | 4 | 4 | 5 |
| A18 | | | | | | | |

TABLE 6

| | Sugar content | | Branched chain | Free arginine |
|---|---|---|---|---|
| | (% by mass) | | fatty acid | content |
| | Glucose | Maltose | mg/100 g | mg/100 g |
| Example A13 | 9.23 | 1.82 | 4.95 | 2.02 |
| Example A14 | 12.43 | 2.25 | 2.3 | 5.112 |
| Example A15 | 10.32 | 1.51 | 3.12 | 3.7 |
| Example A16 | 11.09 | 1.62 | 2.8 | — |
| Example A17 | 9.08 | 1.84 | 1.14 | 0.96 |
| Example A18 | 12.68 | 1.65 | 2.07 | 1.1 |

TABLE 7

| | Raw materials | | | | | | | | | | |
|---|---|---|---|---|---|---|---|---|---|---|---|
| | | | Dietary | | Rice koji | | | | | | |
| | | Starch | fiber | Diameter or | | Starch | | | Bacillus subtilis natto | | |
| | | content | content | major axis | | content | ratio of | Salt | | Number of | Main f [symbol] |
| | | (% by | (% by | length | | (% by | koji:raw | content | Bacteria | inoculation | Tempe [symbol] |
| | Type | mass) | mass) | (mm) | Type | mass) | materials | mg/100 g | strain | (cells/g) | (° C.) |
| Example | Soybean | 0.6 | 21.5 | 8 | Yellow koji | 55.2 | 1:5 | 0 | B-1 | 1000 | 52 |
| A19 | | | | | powder | | | | | | |
| Example | Soybean | 0.6 | 21.5 | 4 | Yellow koji | 55.2 | 1:5 | 0 | B-1 | 1000 | 52 |
| A20 | hikiwari | | | | powder | | | | | | |
| Example | Chickpea | 35.4 | 16.3 | 12 | Yellow koji | 55.2 | 1:5 | 0 | B-1 | 1000 | 52 |
| A21 | | | | | powder | | | | | | |
| Example | Chickpea | 35.4 | 16.3 | 4 | Yellow koji | 55.2 | 1:5 | 0 | B-1 | 1000 | 52 |
| A22 | hikiwari | | | | powder | | | | | | |
| Example | Red bean | 41.7 | 24.8 | 7 | Yellow koji | 55.2 | 1:5 | 0 | B-1 | 1000 | 52 |
| A23 | | | | | powder | | | | | | |
| Example | Peas | 37 | 17.4 | 10 | Yellow koji | 55.2 | 1:5 | 0 | B-1 | 1000 | 52 |
| A24 | | | | | powder | | | | | | |
| Example | Yellow pea | 40.8 | 17.4 | 11 | Yellow koji | 55.2 | 1:5 | 0 | B-1 | 1000 | 52 |
| A25 | | | | | powder | | | | | | |
| Example | Yellow pea | 40.8 | 17.4 | 4 | Yellow koji | 55.2 | 1:5 | 0 | B-1 | 1000 | 52 |
| A26 | hikiwari | | | | powder | | | | | | |
| Example | Red kidney | 35.7 | 19.6 | 17 | Yellow koji | 55.2 | 1:5 | 0 | B-1 | 1000 | 52 |
| A27 | bean | | | | powder | | | | | | |
| Example | White | 35.7 | 19.6 | 17 | Yellow koji | 55.2 | 1:5 | 0 | B-1 | 1000 | 52 |
| A28 | kidney bean | | | | powder | | | | | | |
| Example | Lentils | 40 | 16.7 | 6 | Yellow koji | 55.2 | 1:5 | 0 | B-1 | 1000 | 52 |
| A29 | | | | | powder | | | | | | |
| Example | Mung bean | 39.9 | 14.6 | 5 | Yellow koji | 55.2 | 1:5 | 0 | B-1 | 1000 | 52 |
| A30 | | | | | powder | | | | | | |
| Example | Glutinous | 65.4 | 12.2 | 3 | Yellow koji | 55.2 | 1:5 | 0 | B-1 | 1000 | 52 |
| A31 | wheat | | | | powder | | | | | | |
| Example | Sweet | 24.1 | 2.2 | 20 | Yellow koji | 55.2 | 1:5 | 0 | B-1 | 1000 | 52 |
| A32 | potato | | | | powder | | | | | | |
| Example | Sweet corn | 4 | 9 | 10 | Yellow koji | 55.2 | 1:5 | 0 | B-1 | 1000 | 52 |
| A33 | | | | | powder | | | | | | |
| Example | Green | 2.9 | 21.5 | 15 | Yellow koji | 55.2 | 1:5 | 0 | B-1 | 1000 | 52 |
| A34 | soybean | | | | powder | | | | | | |
| Example | Pumpkin | 8.6 | 2.8 | 20 | Yellow koji | 55.2 | 1:5 | 0 | B-1 | 1000 | 52 |
| A35 | | | | | powder | | | | | | |
| Example | Green pea | 9.1 | 17.4 | 5 | Yellow koji | 55.2 | 1:5 | 0 | B-1 | 1000 | 52 |
| A36 | | | | | powder | | | | | | |
| Example | Ginkgo nut | 29 | 1.6 | 18 | Yellow koji | 55.2 | 1:5 | 0 | B-1 | 1000 | 52 |
| A37 | | | | | powder | | | | | | |

[symbol]

indicates data missing or illegible when filed

TABLE 8

Sugar content (% by mass)

| | Glucose | Maltose |
|---|---|---|
| Example A19 | 9.92 | 0.26 |
| Example A20 | 11.02 | 0.14 |
| Example A21 | 13.4 | 1.76 |
| Example A22 | 15.61 | 1.18 |
| Example A23 | 22.46 | 1.07 |
| Example A24 | 13.63 | 1.85 |
| Example A25 | 15.76 | 1.42 |
| Example A26 | 17.02 | 1.07 |
| Example A27 | 12.73 | 2.42 |
| Example A28 | 12.12 | 1.56 |
| Example A29 | 23.91 | 0.63 |
| Example A30 | 18.5 | 0.35 |
| Example A31 | 25.91 | 2.27 |
| Example A32 | 19.57 | 2.12 |
| Example A33 | 18.55 | 0.69 |
| Example A34 | 11.4 | 0.16 |
| Example A35 | 13.4 | 1.18 |
| Example A36 | 19.53 | 0.67 |
| Example A37 | 11.73 | 0.21 |

### Test Example 2: Fermentation Test Using Chickpea as Raw Material

### Examples a1 to a 15 and Reference Examples b1 to b5; Production of Samples

The samples of Examples al to a15 and Reference Examples b1 to b5 were produced in the following procedure. (However, among the above, Reference Examples b1 and b4 are bean-fermented foods fermented only with koji since Bacillus subtilis natto is not inoculated. Reference Example b3 is not a fermented food because steamed chickpeas themselves are used.)

As the raw material beans, dried chickpeas of a large grain type were used except for Reference Example b5. For Reference Example b5 alone, very small grains of dry soybeans were used.

### (Preparation of Steamed Beans)

Steamed beans used in Examples and Reference Examples were prepared by the following method.

First, dried chickpeas (as described above, in Reference Example b5 alone, very small grains of dried soybean are used) as a raw material were lightly washed with water, and treated in a refrigerator for 18 hours in a state of being immersed in water, so that the water was sufficiently absorbed by the beans, and then the water was drained.

Subsequently, the water-drained immersed beans were subjected to a steaming step. Specifically, the immersed bean was placed in a metal container, placed in a steaming pot (steaming pot for test manufactured by HARADA SANGYO CO., LTD.), heated to a temperature of 98° C., then pressurized under the conditions shown in Table 1 above, and then depressurized to perform steaming.

In Reference Example b3, the steamed beans thus obtained were used as it were, and uninoculated chickpeas were dispensed into a tray.

In Reference Examples b1 and b4, only koji was mixed with the steamed beans thus obtained by the method described later. In Reference Example b2, Bacillus subtilis natto was inoculated into the steamed beans thus obtained by the method described later.

On the other hand, in other Examples and Reference Examples except Reference Examples b1 to b4, koji was mixed with steamed beans immediately after steaming by the method described later to inoculate Bacillus subtilis natto.

### (Fermentation of Natto)

Each of the steamed beans after completion of steaming in the above procedure was inoculated in the following procedure. As Bacillus subtilis K-2 strain (NITE BP-1577) or pure culture Bacillus subtilis natto (Miyagino bacteria) (produced by Miyagino Natto Manufacturing Co., Ltd.) was used. The latter pure culture of Bacillus subtilis natto (Miyagino bacteria) was used only in Example a3.

These Bacillus subtilis natto strains were inoculated into 10 ml/test tube of a spore formation medium (YE) shown in the Table 2 of Test Example 1, and shake-cultured at 37° C. for 200×g for 24 hours to obtain a spore suspension.

As the Bacillus subtilis natto strain used in Examples and Reference Examples, the above-mentioned Bacillus subtilis natto strain was used, and a spore suspension cultured in a liquid medium was diluted with water so that about 1000 Bacillus subtilis natto were present per 1 g of boiled beans, and the resulting mixture was inoculated into steamed beans.

Immediately after inoculation of Bacillus subtilis natto, koji was mixed at a ratio described in Table 9 described later (“Table 9-1 to Table 9-3”, and the same applies hereinafter). As koji, the yellow koji powder (manufactured by Hishiroku Co., Ltd.) was used, and mixed at a ratio shown in Table 9. The steamed beans after being mixed with koji were placed in polystyrene natto containers in the amounts shown in Table 9, respectively, and subjected to fermentation.

However, in Reference Example b1, only koji was mixed with the steamed beans and dispensed into a polystyrene natto container without inoculating Bacillus subtilis natto. Next, in Reference Example b2, only Bacillus subtilis natto was inoculated without using koji and then dispensed into a polystyrene natto container. In Reference Example b3, without using koji or Bacillus subtilis natto, only boiled beans (steamed chickpeas) were dispensed into a polystyrene natto container.

Furthermore, in Reference Example b5, steamed soybeans were used as the steamed beans, and immediately after inoculating the Bacillus subtilis natto, koji was mixed therewith, and then the mixture was dispensed into a polystyrene natto container.

In addition, the coverage of the koji with respect to the surface area of the raw material ((the surface area of the edible plant serving as the raw material to which the powder having an α-amylase activity is attached/the surface area of the edible plant serving as the raw material)×100) was 50% or more.

Furthermore, in Reference Example b4, fermentation was performed according to the following procedure with reference to the production of miso.
1) 112 g of the steamed chickpeas placed in a resin standing pouch (produced by Seinichigrips LZ-16, manufactured by SEISAN NIPPONSHA,Ltd.), and crushed by hand were thoroughly mixed with 112 g of a yellow koji powder (manufactured by Hishiroku Co., Ltd.) in a metal bowl.
2) 50 g of salt was added to the mixture of the chickpeas and koji, and the mixture was uniformly mixed.
3) Further, 100 g of a salt solution obtained by mixing 82 g of water and 18 g of salt was mixed with the mixed powder of chickpea salt and thoroughly kneaded.
4) The above mixture was adjusted into a spherical shape, placed in a standing pouch made of resin (SeinichGrips LZ-16 manufactured by SEISAN NIPPONSHA,Ltd.), flattened while removing air, and an opening of the resin was welded using a vacuum sealer.
5) The mixture in the pouch was placed in an incubator at 37° C. and fermented for 1 month.

The control temperature of the chamber during fermentation for the samples other than Reference Examples b3 and b4 (hereinafter, abbreviated as room temperature) is as shown in Table 9-2. As a fermentation chamber, a natto fermentation chamber (fermentation chamber for test manufactured by HARADA SANGYO CO., LTD.) was used.

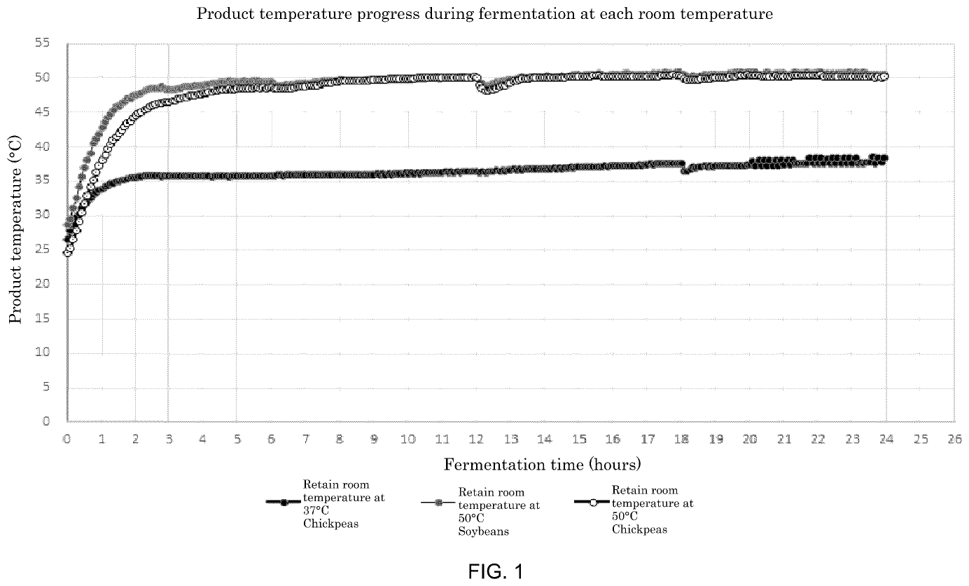

FIG. 1 is a graph showing temporal changes in a control temperature (room temperature) and a product temperature of a fermentation chamber during fermentation of a sample in Examples and Reference Examples other than Reference Examples b3 and b4 as described above.

As shown in FIG. 1, when the room temperature was set to 37° C., the product temperature, which is a normal fermentation temperature zone, was maintained in the range of 35° C. or more and 54° C. or less (more specifically, 35° C. or more and 39° C. or less) after the lapse of 1.5 hours from the start of fermentation. On the other hand, when the room temperature was set to 50° C., the product temperature in the normal fermentation temperature zone was maintained in the range of 35° C. or more and 54° C. or less (more specifically, 35° C. or more and 52° C. or less) after the lapse of 1 hour from the start of fermentation, and the product temperature in the high-temperature fermentation temperature zone was maintained in the range of 45° C. or more and 54° C. or less (more specifically, 45° C. or more and 52° C. or less) after the lapse of 2 hours from the start of fermentation.

For Examples and Reference Examples other than Reference Examples b3 and b4, the fermentation step was performed by maintaining at the room temperature for the time shown in Table 9. For Reference Example b3, the steamed chickpeas themselves were used in the test as described above. For Reference Example b4, fermentation was performed in an incubator set at 37° C. for 1 month.

The sample after completion of the fermentation step was aged by cooling in a refrigerator at 4° C. The quality evaluation was performed on each sample after aging. The analysis results and sensory evaluation of each component are shown in Table 9-3.

(Preparation after Production of Sample)

For Examples a 10 to a14, after a sample was produced by the method described above, glucose, maltose, L-arginine, 4-vinyl-2-methoxyphenol, and 2-phenylethyl alcohol were each added so as to have the concentration described in Table 9-2. When the reagents of these components were added, they were each diluted with ultrapure water to a high concentration and then added. The following standard substances were used for addition.
Glucose (manufactured by KANTO CHEMICAL CO., INC.)
Maltose (manufactured by Sigma-Aldrich Co. LLC.)
L-arginine (manufactured by FUJIFILM Wako Pure Chemical Corporation)
4 vinyl-2-methoxyphenol (CAS No. 7786-61-0) (manufactured by FUJIFILM Wako Pure Chemical Corporation)
2-phenylethyl alcohol (CAS No. 60-12-8) (manufactured by Tokyo Chemical Industry Co., Ltd.)

### <Sensory Evaluation>

Sensory evaluation was performed by the following method.

Evaluation of each sample was performed under the following conditions. Each evaluation was performed by the following three trained sensory inspectors, and a value rounded off to the first decimal place of the average score was described as the score is shown in Table 9 (Table 9-3).

The sensory inspector was selected in the same manner as in Test Example 1.

A sensory test item was “chestnut like flavor”, and evaluation was performed according to the following five-grade criteria.
Evaluation criteria for chestnut like flavor
   5: It is extremely preferable because the chestnut like flavor is clearly felt.
   4: It is preferable because the chestnut like flavor is felt.
   3: It is in an acceptable range because chestnut like flavor is slightly felt.
   2: It is not preferable because the chestnut like flavor is hardly felt.
   1: It is extremely not preferable because the chestnut like flavor is not felt at all.

The results of the sensory test are shown in the following Table 9 (Table 9-3).

Examples generally satisfied the reference value as 3 points or more, but Reference Examples generally satisfied 2 points or less, and did not satisfy the reference value.

In particular, as shown in Examples a 10 to a 15, when the glucose content, the maltose content, the arginine content, the 2-phenylethyl alcohol content, and the 4-vinyl-2-methoxyphenol content are each within a prodetermined range, it is understood that the sensory evaluation score can be 3.5 points or more.

TABLE 9-1

| | | | | | Bacillus | Inoculation amount of |
|---|---|---|---|---|---|---|
| | Koji | Chickpea | Soybean | Ratio of rice | subtilis | Bacillus subtilis natto |
| | g | g | g | koji:chickpea | natto | cells/g |
| Example a1 | 8 | 48 | — | 1:06 | K2 | 1000 |
| Example a2 | 8 | 48 | — | 1:06 | K2 | 1000 |
| Example a3 | 8 | 48 | — | 1:06 | Miyagino | 1000 |
| Example a4 | 16 | 48 | — | 1:03 | K2 | 1000 |
| Example a5 | 4.8 | 48 | — | 1:10 | K2 | 1000 |
| Example a6 | 8 | 48 | — | 1:06 | K2 | 1000 |
| Example a7 | 8 | 48 | — | 1:06 | K2 | 1000 |
| Example a8 | 8 | 48 | — | 1:06 | K2 | 1000 |
| Example a9 | 8 | 48 | — | 1:06 | K2 | 1000 |
| Example a10 | 8 | 48 | — | 1:06 | K2 | 1000 |
| Example a11 | 8 | 48 | — | 1:06 | K2 | 1000 |
| Example a12 | 8 | 48 | — | 1:06 | K2 | 1000 |
| Example a13 | 8 | 48 | — | 1:06 | K2 | 1000 |
| Example a14 | 8 | 48 | — | 1:06 | K2 | 1000 |
| Example a15 | 8 | 48 | — | 1:06 | K2 | 1000 |
| Comparative | 8 | 48 | — | 1:06 | None | None |
| Example b1 | | | | | | |
| Comparative | None | 48 | — | — | K2 | 1000 |
| Example b2 | | | | | | |
| Comparative | None | 48 | — | — | None | None |
| Example b3 | | | | | | |
| Comparative | 112 | 112 | — | 1:01 | None | None |
| Example b4 | | | | | | |
| Comparative | 8 | | 48 | 1:6* | K2 | 1000 cells |
| Example b5 | | | | | | |

TABLE 9-2

| | Fermentation | Time during | | Time during | | | | | | |
|---|---|---|---|---|---|---|---|---|---|---|
| | conditions | which product | | which product | | | | | | |
| | Room | temperature | | temperature | | Addition | Addition | | | Addition |
| | temperature | is maintained | | is maintained | | amount | amount | Addition | Addition | amount of |
| | (° C.) | at normal | | in high- | | of | of | amount | amount of 4- | 2- |
| | maintaining | fermentation | | temperature | | glucose | maltose | of | vinyl-2 | phenylethyl |
| | time | temperature zone | | fermentation zone | | % by | % by | arginine | methoxyphenol | alcohol |
| | (hours) | Time | | Time | | mass | mass | mg/100 g | μg/kg | μg/kg |
| Example a1 | 50° C. 12 h | 11 | h | 10 | h | | | | | |
| Example a2 | 37° C. 18 h | 16.5 | h | — | | | | | | |
| Example a3 | 50° C. 12 h | 11 | h | 10 | h | | | | | |
| Example a4 | 50° C. 12 h | 11 | h | 10 | h | | | | | |
| Example a5 | 50° C. 12 h | 11 | h | 10 | h | | | | | |
| Example a6 | 50° C. 24 h | 23 | h | 22 | h | | | | | |
| Example a7 | 37° C. 24 h | 22.5 | h | — | | | | | | |
| Example a8 | 50° C. 6 h | 5 | h | 4 | h | | | | | |
| Example a9 | 37° C. 12 h | 10.5 | h | — | | | | | | |
| Example a10 | 50° C. 12 h | 11 | h | 10 | h | 6.896 | | | | |
| Example a11 | 50° C. 12 h | 11 | h | 10 | h | | 3.871 | | | |
| Example a12 | 50° C. 12 h | 11 | h | 10 | h | | | 52.856 | | |
| Example a13 | 50° C. 12 h | 11 | h | 10 | h | | | | 84.8 | |
| Example a14 | 50° C. 12 h | 11 | h | 10 | h | | | | | 167.3 |
| Example a15 | 50° C. 18 h | 17 | h | 16 | h | | | | | |
| Comparative | 50° C. 12 h | 11 | h | 10 | h | | | | | |
| Example b1 | | | | | | | | | | |
| Comparative | 50° C. 12 h | 11 | h | 10 | h | | | | | |
| Example b2 | | | | | | | | | | |
| Comparative | 50° C. 12 h | 11 | h | 10 | h | | | | | |
| Example b3 | | | | | | | | | | |
| Comparative | 37° C. 1 | 1 | month | — | | | | | | |
| Example b4 | month | | | | | | | | | |
| Comparative | 50° C. 12 h | 11 | h | 10 | h | | | | | |
| Example b5 | | | | | | | | | | |

TABLE 9-3

| | | Viable | | | | | | Chestnut |
|---|---|---|---|---|---|---|---|---|
| | | cell | Glucose | Maltose | | 2-phenylethyl | 4-vinyl-2 | like |
| | Salt | count | % by | % by | Arginine | alcohol | methoxyphenol | flavor |
| | mg/100 g | cells/g | mass | mass | mg/100 g | μg/kg | μg/kg | Point |
| Example a1 | 5.69 | 5.1 × 10⁷ | 13.1 | 1.1 | 137.1 | 15.2 | 62.7 | 5 |
| Example a2 | 6.61 | 6.4 × 10⁷ | 12.1 | 1.2 | 107.4 | 15.6 | 54.9 | 4 |
| Example a3 | 6.89 | 8.3 × 10⁷ | 12.2 | 1.1 | 152.6 | 24.7 | 78.9 | 5 |
| Example a4 | 6.04 | 4.5 × 10⁶ | 16.2 | 0.7 | 155.8 | 15.5 | 82.1 | 4 |
| Example a5 | 6.45 | 4.5 × 10⁷ | 8.3 | 1.3 | 132.3 | 13.7 | 52.6 | 4 |
| Example a6 | 6.36 | 2.2 × 10⁸ | 12.8 | 1.4 | 125.7 | 28.8 | 53 | 4 |
| Example a7 | 5.93 | 1.1 × 10⁸ | 11.5 | 1.1 | 137.4 | 24 | 45.3 | 4 |
| Example a8 | 6.05 | 4.0 × 10⁵ | 9.7 | 1.4 | 132.2 | 10.8 | 61.8 | 3 |
| Example a9 | 5.93 | 3.5 × 10⁶ | 11.1 | 1.2 | 108.6 | 15.7 | 51.9 | 4 |
| Example a10 | 5.69 | 5.1 × 10⁷ | 20 | 1.1 | 137.1 | 15.2 | 62.7 | 5 |
| Example a11 | 5.69 | 5.1 × 10⁷ | 13.1 | 5 | 137.1 | 15.2 | 62.7 | 4 |
| Example a12 | 5.69 | 5.1 × 10⁷ | 13.1 | 1.1 | 190 | 15.2 | 62.7 | 4 |
| Example a13 | 5.69 | 5.1 × 10⁷ | 13.1 | 1.1 | 137.1 | 100 | 62.7 | 4 |
| Example a14 | 5.69 | 5.1 × 10⁷ | 13.1 | 1.1 | 137.1 | 15.2 | 230 | 4 |
| Example a15 | 6.29 | 1.7 × 10⁸ | 14.5 | 1 | 138.8 | 20.3 | 59.7 | 4 |
| Comparative | 7 | ND | 13.7 | 1 | 197.1 | 3.1 | 6.6 | 2 |
| Example b1 | | | | | | | | |
| Comparative | 7.72 | 2.8 × 10⁸ | 0.6 | 0 | 16.5 | 4.2 | 1.1 | 1 |
| Example b2 | | | | | | | | |
| Comparative | 10.08 | ND | 0.3 | 0 | 100.6 | 1.4 | 1.3 | 1 |
| Example b3 | | | | | | | | |
| Comparative | 14753 | ND | 24.7 | 3.2 | 136.5 | 1.6 | 18.8 | 1 |
| Example b4 | | | | | | | | |
| Comparative | 2.93 | 5.4 × 10⁷ | 11 | 0.4 | 53.1 | 17.7 | 249.2 | 2 |
| Example b5 | | | | | | | | |

### Test Example 3. Test for Examining Particle Size of Koji Powder

### Examples a16 to a 19 and Reference Examples b6 to b7; Production of Samples

The samples of Examples a16 to a19 and Reference Examples b6 to b7 were produced in the following procedure. The koji was mixed with the steamed chickpeas prepared as described above at a ratio described in Table 10 below. The steamed beans after being mixed with koji were placed in polystyrene natto containers in the amounts shown in Table 9, respectively, and subjected to fermentation.

A yellow koji powder of 100 mesh pass in Example a16, 83 mesh pass of an opening size in a17, 50 mesh pass in a18, and 30 mesh pass in a19 was used, and the sizes of the yellow koji powders were equalized. In Reference Example b6, an equal amount of water was added to and suspended in a yellow koji powder to prepare a 50% (w/w) yellow koji aqueous solution. In Reference Example b7, an equal amount of hot water was added to and suspended in the yellow koji powder to prepare a 50% (w/w) yellow koji aqueous solution.

TABLE 10

| | Raw materials | | | | | | | |
|---|---|---|---|---|---|---|---|---|
| | | | Dietary | Diameter | Rice koji | | | |
| | | Starch | fiber | or major | | | Moisture | d50 |
| | | content | content | axis | | | content | (μm) |
| | | (% by | (% by | length | | | (% by | after |
| | Type | mass) | mass) | (mm) | Type | State | mass) | sonication |
| Example | Chickpea | 35.4 | 16.3 | 12 | Yellow | Dry | 2.6 | 79.5 |
| a16 | | | | | koji | powder | | |
| | | | | | powder | | | |
| Example | Chickpea | 35.4 | 16.3 | 12 | Yellow | Dry | 2.6 | 184.5 |
| a17 | | | | | koji | powder | | |
| | | | | | powder | | | |
| Example | Chickpea | 35.4 | 16.3 | 12 | Yellow | Dry | 2.6 | 259.6 |
| a18 | | | | | koji | powder | | |
| | | | | | powder | | | |
| Example | Chickpea | 35.4 | 16.3 | 12 | Yellow | Dry | 2.6 | 484.1 |
| a19 | | | | | koji | powder | | |
| | | | | | powder | | | |
| Reference | Chickpea | 35.4 | 16.3 | 12 | Yellow | 50% | — | — |
| Example | | | | | koji | (w/w) | | |
| b6 | | | | | powder | aqueous | | |
| | | | | | | solution | | |
| Reference | Chickpea | 35.4 | 16.3 | 12 | Yellow | 50% | — | — |
| Example | | | | | koji | (w/w) | | |
| b7 | | | | | powder | aqueous | | |
| | | | | | | solution | | |

| | | Ratio | Main | | 60° C. | | | |
|---|---|---|---|---|---|---|---|---|
| | mesh | of rice | fermentation | | step | Sensory | | |
| | diameter | koji:chick | Temperature | Time | Time | | Natto | |
| | (μm) | pea | (° C.) | (h) | (h) | Sweetness | odor | Texture |
| Example | 150 | 1:6 | 55 | 11 | 1.5 | 5 | 5 | 5 |
| a16 | | | | | | | | |
| Example | 180 | 1:6 | 55 | 11 | 1.5 | 4 | 4 | 5 |
| a17 | | | | | | | | |
| Example | 297 | 1:6 | 55 | 11 | 1.5 | 3 | 3 | 4 |
| a18 | | | | | | | | |
| Example | 500 | 1:6 | 55 | 11 | 1.5 | 3 | 3 | 3 |
| a19 | | | | | | | | |
| Reference | — | 1:2 | 55 | 11 | 1.5 | 1 | 1 | 1 |
| Example | | | | | | | | |
| b6 | | | | | | | | |
| Reference | — | 1:10 | 55 | 11 | 1.5 | 1 | 1 | 1 |
| Example | | | | | | | | |
| b7 | | | | | | | | |

### <Measurement of Particle Size Distribution>

After sonication of the yellow koji powder, d50 was measured as follows.

Measurement was performed with a laser diffraction particle size distribution analyzer (Microtrac MT3300 EXI, manufactured by MicrotracBEL Corp.). Ethanol was used as a solvent at the time of measurement, and DMS2 (DataManagement System version II, manufactured by MicrotracBEL Corp.) was used as measurement application software.

First, the cleaning button of the measurement application software was pressed to clean, then the SetZero button of the same software was pressed to perform zero alignment, and the sample was directly input until the concentration reached the appropriate concentration range at sample loading. Next, the sonication button of the same software was pressed, sonication was performed at 30 kHz, 40 W, for 180 seconds, defoaming treatment was performed 3 times, and laser diffraction was performed at a flow rate of 50% for a measurement time of 10 seconds, and the result was taken as a measurement value. The measurement conditions are as follows: distribution display: volume; particle refractive index: 1.60; solvent refractive index: 1.36; measurement upper limit (μm): 2000; and the lower limit of measurement (μm) were set to 0.021.

### <Measurement of Dry Basis Moisture Content of Koji>

In accordance with the present STANDARD TABLES OF FOOD COMPOSITION—2020—(Eighth Revised Edition), it was measured by heating to 90° C. by a vacuum heat drying method. Specifically, an appropriate amount of sample was collected and weighed in a scale container (W0) having a constant mass in advance (W1), placed in a reduced pressure electric constant temperature dryer adjusted to a predetermined temperature (more specifically,90° C.) at normal pressure in a state where the mouth of the scale container was opened, the door was closed, the vacuum pump was operated, drying was performed at a predetermined degree of pressure reduction for a certain period of time, the vacuum pump was stopped, the pressure is returned to normal pressure by sending dry air, the scale container was taken out, the lid was closed, and the sample was cooled in a desiccator, and then the mass was measured. In this way, drying, cooling, and weighing (W2) were repeated until the weight reached a constant mass, and the dry weight standard water content (dry basis moisture content) (% by mass) was determined by the following calculation formula.

$$Water content ( dry basis moisture content ) ( \% by mass ) = ( {W}_{1} - {W}_{2} ) / ( {W}_{2} - {W}_{0} ) \times 100 \qquad [ Mathematical formula 3 ]$$

(In the formula, W₀ is the mass (g) of the scale container with constant mass, W₁ is the mass (g) of the scale container containing the sample before drying, and W2 is the mass (g) of the scale container containing the sample after drying.)

### <Sensory Evaluation>

Sensory evaluation was performed in the same manner as in Test Example 1.

The results of the particle size distribution measurement and sensory evaluation are shown in Table 10. The reference value was satisfied with 3 points or more in Examples, but the reference value was not satisfied with 2 points or less in Reference Examples. It is considered that when koji is added as a liquid, the koji mold does not enter the inside of the raw material, and the sweetness of the fermented food is insufficient.

### <Variation in Starch Content>

The starch content before and after fermentation was measured by the following method.

As the starch content before the water absorption treatment, a value measured by AOAC method 996.11 (AOAC, 2005) was used, in accordance with the method in STANDARD TABLES OF FOOD COMPOSITION IN JAPAN—2020—(Eighth Revised Edition). Steamed chickpeas were prepared in the same manner as in Example a2, and the starch content was measured in the same manner. A yellow koji powder or Bacillus subtilis natto was inoculated according to the formulation shown in Table 11, and the starch content was measured in the same manner. Fermentation was performed under the conditions described in Table 11, the fermentation start time was set to 0 hour, sampling was performed at 0 hour, 6 hours, 12 hours, and 18 hours after the start of fermentation, and the starch content was measured in the same manner. The results are shown in Table 11.

TABLE 11

| Raw materials | | | | | | | | | | | | | |
|---|---|---|---|---|---|---|---|---|---|---|---|---|---|
| | Starch content | | | | | | | | | | | | |
| | (% by mass) | | Koji | | | Bacillus subtilis | | Total | | Starch content | | | |
| | Before | | | Starch | | natto | | starch | | (% by mass) | | | |
| | water | | | content | ratio of | | Number of | content | Fermentation | Fermentation | | | |
| | absorption | After | | (% by | koji:raw | | inoculation | (% by | temperature | time | | | |
| Type | treatment | steaming | Type | mass) | materials | Type | (cells/g) | mass) | (° C.) | 0 h | 6 h | 12 h | 18 h |
| Chickpea | 36.9 | 20 | None | None | 1:6 | K2 | 1000 | 20 | 37 | 20 | — | — | 17.8 |
| Chickpea | 36.9 | 20 | Yellow koji | 55.2 | 1:6 | None | 0 | 25 | 37 | 25 | 13.7 | 13 | 10 |
| | | | powder | | | | | | | | | | |
| Chickpea | 36.9 | 20 | Yellow koji | 55.2 | 1:6 | K2 | 1000 | 25 | 37 | 25 | 12.6 | 12.1 | 11.1 |
| | | | powder | | | | | | | | | | |

### <Enzyme Activity of Koji>

The acid protease activity was measured according to the National Tax Agency predetermined analysis method (The Japan Brewing Society, Commentary on the 4th revised National Tax Agency predetermined analysis method, p. 223, 2003). The α-amylase activity was measured by the following procedure.

### Preparation of Enzyme Liquid:

First, 10 mL of 0.5% NaCl/10 mM acetic acid buffer (pH 5) was added to 1 g of the measurement sample obtained by pulverizing the compositions of each test section and comparative section, and the mixture was allowed to stand at 4° C. for 16 hours, then the mixture was shattered into a paste by treatment at 25000 rpm for 30 seconds using a homogenizer NS52 (manufactured by MICROTEC CO., LTD.), further allowed to stand at 4° C. for 16 hours, and then filtered with a filter paper (Qualitative filter paper No. 2, manufactured by ADVANTEC Corporation) to obtain an enzyme liquid.

In a test tube, 2 mL of a 0.05% soluble starch (starch (soluble) CAS No. 9005-25-8, product code 195—03961, manufactured by FUJIFILM Wako Pure Chemical Corporation) was put, and the resulting mixture was allowed to stand at 37° C. for 10 minutes. Then, 0.25 mL of the enzyme liquid was added, and the resulting mixture was mixed. The mixture was allowed to stand at 37° C. for 30 minutes. Thereafter, 0.25 mL of 1 M HCl was added and mixed, 0.25 mL of a 0.05 mol/L iodine solution was added and mixed, 11.5 mL of water was added and diluted, and the absorbance at a wavelength of 660 nm was measured with a spectrophotometer (absorbance A). In addition, as a control, 2 mL of a 0.05% soluble starch was placed in a test tube, left to stand at 37° C. for 40 minutes, then 0.25 mL of 1 M HCl was added and mixed, then 0.25 mL of an enzyme liquid and 0.25 mL of a 0.05 mol/L iodine solution were added in this order, and the mixture was diluted by adding 11.5 mL of water, and then the absorbance at a wavelength of 660 nm was measured with a spectrophotometer (absorbance B).

The absorbance reduction rate C (%) of each measurement sample during the enzymatic reaction for 30 minutes was determined from the absorbance reduction rate of the enzyme reaction section (absorbance A) with respect to the comparative target section (absorbance B), that is, ({(absorbance B−absorbance AVabsorbance B}×100(%)). The enzyme activity (U/g) for reducing the absorbance by 10% per 10 minutes was defined as 1 unit (U), and the enzyme activity per 1 g of dry mass of the measurement sample was determined from the absorbance reduction rate C (%) when the enzyme reaction was carried out for 30 minutes with a 0.25 mL of an enzyme liquid (sample content: 0.025 g) by the following formula.

$$Enzyme activity unit ( U / g ) = \{ C \times ( 10 / 30 ) \times ( 1 / 10 ) \} / 0.025 \qquad [ Mathematical formula 3 ]$$

As a result, the α-amylase activity and the acidic protease activity of the yellow koji powder used in the above Test Examples were 1140 U/g and 4731 U/g, respectively.

### Test Example 4. Examination of Koji Powder

### Examples a20 and a21; Production of Samples

The sample of Example a20 was produced in the same procedure as in Example A18 except that koji was changed to rice koji B (manufactured by KOHSEI FOODS CO., LTD.). The sample of Example a21 was produced in the same procedure as in Example A4, except that the koji was changed to white koji powder (koji mold: Aspergillus Kawachii, manufactured by Hishiroku Co., Ltd.). The formulation and the like are described in Table 12. The steamed beans after being mixed with koji were placed in polystyrene natto containers in the amounts shown in Table 9, respectively, and subjected to fermentation.

<Koji B, Dry Basis Moisture Content of White Koji Powder, d50>

In the same manner as in Test Example 3, the dry basis moisture content and d50 of rice koji B and a white koji powder were measured. In each case, the dry basis moisture content was 10% by mass or less, and d50 after sonication was 500 μm or less.

### <Enzyme Activity of Koji>

The enzyme activity of the koji was measured in the same manner as in Test Example 3. As a result, the α-amylase activity of the white koji powder was 1140 U/g, and the acidic protease activity thereof was 4731 U/g. The rice koji B had an α-amylase activity of 1345 U/g and an acidic protease activity of 11424 U/g.

### <Analysis of Sugar Composition>

The analysis of the sugar composition was performed in the same manner as in Test Example 1.

### <Sensory Evaluation>

Sensory evaluation was performed in the same manner as in Test Example 1.

The results of the sensory evaluation are shown in Table 12, and the analysis results of the sugar composition are shown in Table 13.

TABLE 12

| | Raw materials | | | | | | | | Bacillus subtilis | |
|---|---|---|---|---|---|---|---|---|---|---|
| | | | Dietary | Diameter | Rice koji | | | | natto | |
| | | Starch | fiber | or major | | Starch | | | | Number |
| | | content | content | axis | | content | ratio of | Salt | | of |
| | | (% by | (% by | length | | (% by | koji:raw | content | Bacteria | inoculation |
| | Type | mass) | mass) | (mm) | Type | mass) | materials | mg/100 g | strain | (cells/g |
| Example | Chickpea | 35.4 | 21.5 | 4 | Rice | 55.2 | 1:6 | 0 | B-1 | 1000 |
| a20 | | | | | koji B | | | | | |
| Example | Yellow | 40.8 | 17.4 | 11 | White | 55.2 | 1:6 | 0 | B-1 | 1000 |
| a21 | pea | | | | koj | | | | | |
| | | | | | powder | | | | | |

| | | | | Viable cell | | | |
|---|---|---|---|---|---|---|---|
| | | | | count of | | | |
| | | | | Bacillus | | | |
| | Main | | 60° C. | subtilis | | | |
| | fermentation | | step | natto | Sensory | | |
| | Temperature | Time | Time | (1000 | | Natto | |
| | (° C.) | (h) | (h) | cells/g) | Sweetness | odor | Texture |
| Example | 55 | 11 | 1.5 | 1,000,000 | 5.0 | 5.0 | 5.0 |
| a20 | | | | | | | |
| Example | 55 | 11 | 1.5 | 1,000,000 | 5.0 | 5.0 | 5.0 |
| a21 | | | | | | | |

TABLE 13

Sugar content
(% by mass)

| | Glucose | Maltose |
|---|---|---|
| Example a20 | 11.6 | 1.3 |
| Example a21 | 13.2 | 5.6 |

### Test Example 5. Heat-Retaining Test of Koji Powder

### Examples a22 to a28, Reference Examples b8 to b10, Production of Samples

The samples of Examples a22 to a28 and Reference Example 8 to 10 were produced in the following procedure. Production was carried out in the same manner as in Example A18, except that heat-retained koji under the conditions described in Table 14 below was used.

### <Analysis of Sugar Composition>

The analysis of the sugar composition was performed in the same manner as in Test Example 1.

### <Sensory Evaluation>

Evaluation of each sample was performed under the following conditions. Each evaluation was performed by the following three trained sensory inspectors, and a value rounded off to the first decimal place of the average score was described as the score is shown in Table 14.

The sensory inspector was selected in the same manner as in Test Example 1.

The item of sensory test was a “dry mealiness mouthfeel”, and evaluation was performed according to the following five-grade criteria.

### · Evaluation Criteria for Mouthfeel

5: It is extremely preferable because dry mealiness is not felt at all.
4: It is preferable because dry mealiness is not felt.
3: It is in an acceptable range because dry mealiness is slightly felt.
2: It is not preferable because dry mealiness is felt.
1: It is extremely not preferable because very dry mealiness is felt.

The results of the sensory evaluation are shown in Table 14, and the analysis results of the sugar composition are shown in Table 15. The reference value was satisfied with 3 points or more in Examples, but the reference value was not satisfied with 2 points or less in Reference Examples. It is considered that when koji is heat retained for a long period of time, the amount of soluble carbohydrates produced decreases, and the mouthfeel of the fermented food becomes dry mealiness.

TABLE 14

| | Raw | | | | | | |
|---|---|---|---|---|---|---|---|
| | materials | | Koji | | | | |
| | | Starch | | Starch | | Heat- | Heat- |
| | | content | | content | ratio of | retaining | retaining |
| | | % by | | % by | koji:raw | temperature | time |
| | Type | mass | Type | mass | materials | ° C. | Time |
| Example | Chickpea | 20 | Yellow koji | 55.2 | 1:6 | 4 | 72 |
| a22 | | | powder | | | | |
| Example | Chickpea | 20 | Yellow koji | 55.2 | 1:6 | 20 | 3 |
| a23 | | | powder | | | | |
| Example | Chickpea | 20 | Yellow koji | 55.2 | 1:6 | 20 | 4 |
| a24 | | | powder | | | | |
| Example | Chickpea | 20 | Yellow koji | 55.2 | 1:6 | 20 | 5 |
| a25 | | | powder | | | | |
| Example | Chickpea | 20 | Yellow koji | 55.2 | 1:6 | 20 | 168 |
| a26 | | | powder | | | | |
| Example | Chickpea | 20 | Yellow koji | 55.2 | 1:6 | 30 | 168 |
| a27 | | | powder | | | | |
| Example | Chickpea | 20 | Yellow koji | 55.2 | 1:6 | 40 | 168 |
| a28 | | | powder | | | | |
| Reference | Chickpea | 20 | Yellow koji | 55.2 | 1:6 | 20 | 720 |
| Example | | | powder | | | | |
| b8 | | | | | | | |
| Reference | Chickpea | 20 | Yellow koji | 55.2 | 1:6 | 30 | 720 |
| Example | | | powder | | | | |
| b9 | | | | | | | |
| Reference | Chickpea | 20 | Yellow koji | 55.2 | 1:6 | 40 | 720 |
| Example | | | powder | | | | |
| b10 | | | | | | | |

| | | | | | | Sensory |
|---|---|---|---|---|---|---|
| | Bacillus subtilis | | Main | | | evaluation |
| | natto | | fermentation | | 60° C. | Dry |
| | | Number of | Fermentation | | step | mealiness |
| | | inoculation | temperature | Fermentation | Time | mouthfeel |
| | Type | cells/g | ° C. | time | Time | Comment |
| Example | B-1 | 1000 | 55 | 11 | 1.5 | 5 |
| a22 | | | | | | |
| Example | B-1 | 1000 | 55 | 11 | 1.5 | 5 |
| a23 | | | | | | |
| Example | B-1 | 1000 | 55 | 11 | 1.5 | 5 |
| a24 | | | | | | |
| Example | B-1 | 1000 | 55 | 11 | 1.5 | 5 |
| a25 | | | | | | |
| Example | B-1 | 1000 | 55 | 11 | 1.5 | 5 |
| a26 | | | | | | |
| Example | B-1 | 1000 | 55 | 11 | 1.5 | 4 |
| a27 | | | | | | |
| Example | B-1 | 1000 | 55 | 11 | 1.5 | 4 |
| a28 | | | | | | |
| Reference | B-1 | 1000 | 55 | 11 | 1.5 | 1 |
| Example | | | | | | |
| b8 | | | | | | |
| Reference | B-1 | 1000 | 55 | 11 | 1.5 | 1 |
| Example | | | | | | |
| b9 | | | | | | |
| Reference | B-1 | 1000 | 55 | 11 | 1.5 | 1 |
| Example | | | | | | |
| b10 | | | | | | |

TABLE 15

Sugar content (% by mass)

| | Glucose | Maltose |
|---|---|---|
| | % by mass | % by mass |
| Example a22 | 12.78 | 1.18 |
| Example a23 | 11.63 | 1.12 |
| Example a24 | 12.41 | 0.88 |
| Example a25 | 11.56 | 1.38 |
| Example a26 | 11.82 | 1.17 |
| Example a27 | 9.59 | 1.38 |
| Example a28 | 9.73 | 1.30 |
| Reference Example b8 | 3.21 | 3.22 |
| Reference Example b9 | 3.12 | 3.12 |
| Reference Example b10 | 2.32 | 2.95 |

1. A fermented food
wherein the fermented food satisfies the following requirements:
(a) a solid raw material is one or more kinds of edible plants selected from the group consisting of beans, cereals, vegetables, and nuts and seeds;
(b) a salt content is 1000 mg or less per 100 g of the fermented food; and
(c) comprises the solid raw material and a koji powder having α-amylase activity.

2.-15. (canceled)

16. The fermented food according to claim 1, further satisfying the requirements that:
the content of soluble carbohydrate is 5% by mass or more and 30% by mass or less.

17. The fermented food according to claim 1, wherein the edible plants of (a) are fermented with koji and Bacillus subtilis natto.

18. (canceled)

19. A method for producing a fermented food by adding a koji powder having an α-amylase activity to a solid raw material, the method comprising all of the following steps (I) to (III):
(I) a step of adjusting a composition satisfying the following conditions:
(a) the raw material is one or more kinds of edible plants selected from the group consisting of beans, cereals, vegetables, and nuts and seeds; and
(b) a salt content is 1000 mg or less per 100 g of the fermented food,
(II) a step of adding the koji powder having α-amylase activity, and optionally Bacillus subtilis natto, to the composition of (I), and
(III) a step of fermenting the composition of (II) at a product temperature of 30° C. or more and 60ºC or less for 5 hours or more and 23 hours or less.

20.-21. (canceled)

22. The method according to claim 19, further satisfying the requirements that:
the method comprises a step of adding Bacillus subtilis natto a mixture, after obtaining the mixture by mixing the composition of (I) and koji in the step (II).

23. The method according to claim 19, further satisfying the requirements that:
the method comprises a step of adding koji to a mixture, after obtaining the mixture by mixing the composition of (I) and Bacillus subtilis natto in the step (II).

24. (canceled)

25. The method according to claim 19, further comprising the following step (IV):
(IV) a step of heating a product to a temperature higher than an average fermentation temperature of the step (III), then performing fermentation at a product temperature of 55° C. or more and 62° C. or less for 0.5 hours or more and 3 hours or less after the step (III).

26. The method according to claim 19, wherein in the step (II), a mass ratio of the koji powder having α-amylase activity to the edible plants of (a) is 1:3 or more.

27. The method according to claim 19, further comprising the following step (V):
(V) a step of aging at a low temperature of 3° C. or more and less than 10ºC for 6 hours to 3 days after completion of fermentation.

28. The method according to claim 19, further satisfying the requirements that:
the koji powder having α-amylase activity in the step (II) has a dry basis moisture content of 15% by mass or less.

29. The method according to claim 19, further satisfying the requirements that:
d50 of the koji powder having α-amylase activity in the step (II) after sonication is 1000 μm or less.

30. The method according to claim 19, further satisfying the requirements that:
the method comprises a step of retaining the koji powder having α-amylase activity in the step (II) at 40° C. or less for 1 hour or more, before adding the koji powder to the composition of (I).

31. The method according to claim 19, further satisfying the requirements that:
the method comprises a step of heat-treating the edible plants of (a) in the step (I) at 80° C. or more under the condition of a dry basis moisture content of 50% by mass or more, by immersing the edible plants of (a) in water for 6 to 24 hours, and then steaming with steam at 100 to 135° C. for 5 to 30 minutes, or by immersing the edible plants of (a) in water for 6 to 24 hours, and then boiling in liquid at 90 to 100° C. for 20 to 50 minutes.

32.-33. (canceled)

34. The method according to claim 19, further satisfying the requirements that:
the α-amylase activity of the koji powder having α-amylase activity is 100 U/g or more in the step (II).

35. The method according to claim 19, further satisfying the requirements that:
a starch content in the edible plants of (a) in the step (I) is 4% by mass or more.

36. The method according to claim 19, further satisfying the requirements that:
the method comprises a step of adjusting a dry basis moisture content of the edible plants of (a) to 40% by mass or more, by immersing the edible plants of (a) in water for 6 to 24 hours in the step (I).

37. The method according to claim 31, further satisfying the requirements that:
the method comprises a step of adjusting a dry basis moisture content of the edible plants of (a) to 40% by mass or more, by the immersing and the steaming or the immersing and the boiling of claim 31.

38. The method according to claim 19, further satisfying the requirements that:
in the step (II), a ratio of a dry basis moisture content of the koji powder to a dry basis moisture content of the edible plants of (a) (the dry basis moisture content of the koji powder/the dry basis moisture content of the edible plants) is 0.001 or more.

39. The method according to claim 19, wherein the beans are one or more kinds of beans selected from the group consisting of Pisum, Cicer, and Phaseolus.

40. A method for producing a fermented food by adding a koji powder having an α-amylase activity to a solid raw material, the method comprising all of the following steps (I) to (V):
(I) a step of adjusting a composition satisfying the following conditions:
(a) the raw material is one or more kinds of edible plants selected from the group consisting of Pisum, Cicer, and Phaseolus;
(b) a salt content is 1000 mg or less per 100 g of the fermented food;
(c) a starch content in the edible plants of (a) is 4% by mass or more;
(d) the koji is Aspergillus oryzae or Aspergillus kawachii;
(e) the α-amylase activity of the koji powder having α-amylase activity is 100 U/g or more; and
(f) the koji powder having α-amylase activity is a koji powder pulverized to d50 after sonication of 1000 μm or less, after koji-made,
(II) a step of adding a koji powder having α-amylase activity and Bacillus subtilis natto, to the composition of (I), to comprise the following steps:
(g) a step of heat-treating the edible plants of (a) in the step (I) at 80° C. or more under the condition of a dry basis moisture content of 50% by mass or more, by immersing the edible plants of (a) in water for 6 to 24 hours, and then steaming with steam at 100 to 135° C. for 5 to 30 minutes, or by immersing the edible plants of (a) in water for 6 to 24 hours, and then boiling in liquid at 90 to 100° C. for 20 to 50 minutes;
(h) a step of adjusting a dry basis moisture content of the edible plants of (a) to 40% by mass or more, by the immersing and the steaming or the immersing and the boiling of the step (g);
(i) a step of adding Bacillus subtilis natto to the composition of (I), after the step (h);
(j) a step of retaining the koji powder having an α-amylase activity at 40° C. or less for 1 hour or more, before adding to the composition of (I); and
(k) a step of mixing the koji powder having an α-amylase activity to satisfy the following requirements, after the step (i), (j),
(1) a mass ratio of the koji powder having α-amylase activity to the edible plants of (a) is 1:3 or more,
(2) a ratio of a dry basis moisture content of the koji powder to a dry basis moisture content of the edible plants of (a) (the dry basis moisture content of the koji powder/the dry basis moisture content of the edible plants) is 0.001 or more, and
(3) mixing the koji powder having α-amylase activity having a dry basis moisture content of 15% by mass or less to the composition of (I),
(III) a step of fermenting the composition of (II) at a product temperature of 30° C. or more and 60° C. or less for 5 hours or more and 23 hours or less,
(IV) a step of heating a product to a temperature higher than an average fermentation temperature of the step (III), then performing fermentation at a product temperature of 55° C. or more and 62° C. or less for 0.5 hours or more and 3 hours or less after the step (III), and
(V) a step of aging at a low temperature of 3° C. or more and less than 10° C. for 6 hours to 3 days after completion of fermentation.
